(19) United States
(12) Patent Application Publication
(10) Pub. No.: US 2005/0096265 A2
(43) Pub. Date: May 5, 2005

(54) CHARACTERIZATION OF A MEMBRANE ESTROGEN RECEPTOR

(75) Inventors: Darlene Deecher, Quakertown, PA (US); Pamela Swiggard, Phoenixville, PA (US); Donald Frail, Wildwood, MO (US); Lawrence O'Connor, Des Plaines, IL (US)

Correspondence Address: DARBY & DARBY P.C., P.O. BOX 5257, NEW YORK, NY, 10150-5257, US

(73) Assignee: Wyeth, Madison, NJ (US)

(21) Appl. No.: 10/480,854

(22) Filed: Feb. 9, 2004

Related U.S. Application Data
(60) Provisional application No. 60/298,499, filed on Jun. 15, 2001.
(60) Provisional application No. 60/350,766, filed on Nov. 13, 2001.

(51) Int. Cl. A61K 38/17; C07K 14/72; C07K 16/28; C07H 21/04

(52) U.S. Cl. 514/12; 530/350; 530/388.22; 435/69.1; 435/320.1; 435/325; 536/23.5

(57) ABSTRACT

Abstract of the Disclosure

The present invention discloses the identification of a novel membrane associated estrogen receptor, termed mER. The membrane associated receptor is involved in rapid signal transduction. Amino acid sequences, nucleic acid sequences, vectors, and host cells are also discussed. Additionally, methods of detecting agonists and antagonists for the receptor are disclosed herein.

### Detailed Description of the Invention

### FIELD OF THE INVENTION

The present invention discloses the identification of a novel estrogen receptor termed mER.

### BACKGROUND OF THE INVENTION

The physiological response to steroid hormones is proposed to be mediated by specific interaction of steroids with nuclear receptors. These receptors are part of a larger family of ligand-activated transcription factors that regulate the expression of target genes. Two different nuclear estrogen receptors have been identified to date and they are designated ERα and ERβ. These receptors consist (in an aminoterminal-to-carboxyterminal direction) of a hypervariable aminoterminal domain that contributes to the transactivation function; a highly conserved DNA-binding domain responsible for receptor dimerization and specific DNA binding; and a carboxyterminal domain involved in ligand-binding, nuclear localization, and ligand-dependent transactivation.

Recently, estrogen and estrogen compounds have also been shown to induce very rapid changes in physiological activity in certain cell types. These changes can occur within minutes and therefore cannot be mediated through the classical genomic mechanism that causes changes in gene transcription. Rapid responses to estrogen are thought to be mediated via a non-genomic mechanism that can include stimulation of nitric oxide production in pulmonary endothelial cells (Russell et al. Proc. Natl. Acad. Sci. U.S.A., 97, 5930, 2000), and increased activation of mitogen-activated protein kinase in neuronal cells (Singer et al., Journal of Neuroscience, 19, 2455, 1999), osteoblasts (Kousteni, et al., Cell, 104, 719, 2001), and breast cancer cells (Razandi et al., Molecular Endocrinology, 14, 1434, 2000).

The genomic effects of estrogen and estrogen compounds is mediated through the estrogen receptor (ER) complex (ER receptor and ligand) which binds to DNA, triggering mRNA synthesis and subsequently, protein synthesis. Little, however, is known about the molecular basis of the non-genomic actions of estrogen and estrogen compounds. This diversity of effects can only partially be explained by our current understanding of ER structure and function. Previous models of ER interactions can be used to understand the slower, genomic signaling pathways by estrogen. However, these models fail to explain the rapid signaling effects now reported for the ER complex. These rapid effects of estrogens do not fit the classic concept of nuclear localization and genomic regulation by the ER complex. However, in some systems, activation of estrogen-induced signaling pathways can be blocked by the same synthetic ER antagonists that block transcriptional activation by classical ER (Aronica, et al., Proc. Natl. Acad. Sci. U.S.A., 91, 8517, 1994).

It has been suggested that the non-genomic actions of estrogen may be mediated by a plasma membrane estrogen receptor (mER). Membrane binding sites for 17-β-estradiol (E2) have been identified in several areas such as the brain, uterus, and liver; and various signal pathways have been implicated.

### SUMMARY OF THE INVENTION

The present invention contemplates an isolated membrane estrogen receptor polypeptide, which membrane estrogen receptor polypeptide is present in a cellular P2 fraction, binds to an antibody specific for a nuclear ERα receptor antibody and binds specifically to an estrogen compound. In an embodiment, the receptor polypeptide is recognized by each of antibodies ER21, H-184, H222, and MC-20. In yet another embodiment, the estrogen compound is 17-β-estradiol or diethylstilbestrol. In one embodiment, the antibody is selected from the group consisting of ER21, H-184, H222, and MC-20. In an additional embodiment, binding of an estrogen compound to the receptor modulates calcium mobilization. In another embodiment, the membrane estrogen receptor polypeptide or a fragment thereof has an apparent molecular weight of 67 kDa as determined by SDS-PAGE. Additionally, the present invention contemplates the receptor polypeptide wherein the polypeptide is not recognized by the ERα receptor antibody SRA1000. In a further embodiment, the membrane estrogen receptor polypeptide is not present in the cellular S2 fraction.

The present invention also contemplates an isolated membrane estrogen receptor polypeptide, which membrane estrogen receptor polypeptide is present in a cellular P2 fraction, binds to the nuclear ERα receptor antibodies ER21, H-184, H222, and MC-20, binds specifically to an estrogen compound, has an apparent molecular weight of 67 kDa, is not recognized by the nuclear ERα receptor antibody SRA1000 and is not present in the cellular S2 fraction.

The present invention also contemplates a method for detecting a membrane estrogen receptor polypeptide, which method comprises detecting binding of a nuclear ERα receptor antibody to a polypeptide present in a membrane of a cell. In one embodiment, the membrane estrogen receptor polypeptide is detected in the P2 cellular fraction. In another embodiment, the membrane estrogen receptor polypeptide is detected in an intact cell. In yet another embodiment, the nuclear ERα receptor antibody is selected from the group consisting of ER21, H-184, H222, and MC-20.

The present invention further contemplates a method for detecting a membrane estrogen receptor polypeptide, wherein the polypeptide is detected upon binding of an estrogen compound to a polypeptide in a sample containing the P2 cellular fraction. In one embodiment, the estrogen compound is 17-β-estradiol or diethylstilbestrol.

The present invention further contemplates a method for identifying a compound that binds the membrane estrogen receptor polypeptide, which method comprises detecting binding of a test compound contacted with a cellular P2 fraction wherein binding of the test compound indicates that the test compound binds to the membrane estrogen receptor. In one embodiment, detection of binding of the test compound comprises detecting inhibition of binding of an estrogen compound to the cellular P2 fraction.

The present invention also contemplates a method for identifying a compound that modulates a membrane estrogen receptor polypeptide, which method comprises detecting calcium mobilization in a cell comprising a membrane estrogen receptor polypeptide contacted with a test compound. In one embodiment, the method for identifying a compound that modulates the polypeptide comprises detecting genomic estrogen receptor activity wherein alteration of genomic activity in the presence of the test compound indicates that the compound does not selectively modulate the polypeptide.

The present invention also contemplates a method of screening for an antagonist of a membrane estrogen receptor polypeptide, which method comprises (i) contacting a cell that expresses the polypeptide with a test compound and an estrogen compound and (ii) detecting decreased calcium mobilization compared to contacting the cell with the estrogen compound alone.

### BRIEF DESCRIPTION OF THE DRAWINGS

Figures 1A-D. Characterization of a rat hypothalamic cell line (D12) A. Predominant phenotype Acobblestone matrix@ indicative of endothelial cells. B. Immunocytochemistry using Von Willebrand factor 8; indicative of endothelial cells. C. Fluorescent labeling of D12 cells with Dil-Ac-LDL; indicative of endothelial cells. D. Immunocytochemistry using Neurofilament M; indicative of neurons

Figures 2A-B. A. Radioligand binding analyses of D12 cytosolic (S2) and membrane (P2) fractions reveal specific E2 labeling. B. Western blot analyses with a commercial ERα antibody (SRA1000, StressGen) indicates that binding activity in P2 preparations is not due to contamination with soluble nuclear ER found in S2. The arrow to the right of the blot indicates the position of ERα and the asterisk denotes an unknown protein that cross-reacts with SRA1000.

Figure 3. Scatchard analysis of saturation binding studies. The mER has similar binding affinity but lower expression levels than ER. Values from parallel Scatchard analyses of S2 or P2 extracts reveal that ER and the mER have similar binding affinities (K_{D}) for the radioligand [¹²⁵I]16α-E2 but are expressed at much different levels (Bₘₐₓ) in D12 cells.

Figures 4A-D. Pharmacological characterization of ER and mER in competition studies indicate they have differing affinities (IC50=s) for various E2 ligands. A and B. Representative binding curves are shown demonstrating ligands with similar binding affinities for ERα and mER (A: 16α-iodoE2; B: estrone). C and D. Representative binding curves are shown demonstrating ligands with dissimilar binding affinities for ERα and mER (C: ICI-182780; D: Raloxifene).

Figures 5A-C. A. Schematic of ERα protein indicating relative locations of epitopes to which the various ERα antibodies were generated. Functional domains of ERα are also depicted including transactivation-1 domain (B), DNA-binding domain (C), hinge region (D), and ligand binding/transactivation-2 domain (E). B and C. Western blot analyses suggest that ER and mER are similar but not identical in amino acid sequence. S2 and P2 extracts (n=3) were probed with various antibodies against different regions of ERα. While all antibodies recognized ERα in S2 extracts (B and C), a subset (MC-20, H222, and ER21) also reacted with a membrane protein in P2 extracts of similar molecular mass (67 kDa) as ERα (C).

Figures 6A-B. Pharmacology of mER is altered in presence of ERα antibody. A. Histogram depicting radioligand binding analyses of S2 and P2 extracts when incubated with either MC-20, SRA1000, or normal IgG control antibodies. Antibodies showed no effect on specific binding in S2 extracts while increased binding was seen in P2 extracts incubated with MC-20. Neither SRA1000 nor normal control IgG showed any effect. B. Histogram of binding analyses performed with increasing amounts of MC-20 antibody demonstrating that effects are dose-dependent.

Figure 7A-B. Immunocytochemical fluorescent staining of D12 cells with antibodies against caveolin-1 and ERα (MC20) confirm the membrane localization of ER. Cells were processed in a manner designed to preserve plasma membrane integrity and therefore minimize nuclear staining for ERα.

Figure 8. Rapid calcium changes are noted in the presence of 100 nM E2. Real-time representation of E2-stimulated [Ca²⁺]ᵢ from FURA 2 A/M loaded D12 cells. E2 was administered 2 min after baseline establishment and change in [Ca²⁺]ᵢ was calculated based on Rmax (ionomycin, 100 nM) and Rmin (EGTA 2 mM) from calibration run.

### DETAILED DESCRIPTION OF THE INVENTION

The present invention is based, in part, on discovery of a novel steroid receptor, which has been termed mER. Preferably, the receptor binds estrogen over other known steroids such as, but not limited to, progesterone and testosterone. The novel receptor is associated with the plasma membrane of the cell, whereas prior estrogen receptors are nuclear. The new estrogen receptor was isolated from the rat hypothalamic cell line D12. D12 cells plated on glass coverslips and incubated with FURA2 A/M were treated with test compounds and regulation of calcium mobilization was determined. Receptor mediated effects on nitric oxide synthase, inositol phosphate formation, and cAMP formation are further suggested.

Saturation binding studies indicated the presence of a single saturable estrogen binding site in the plasma membrane, that was recognized by [¹²⁵I]-16α-iodo-3,17β-E2. Scatchard analysis indicated a K_{D} of 118 pM and Bₘₐₓ of 32 fmol/mg protein. Comparison to preparations of the nuclear ERα receptor, showed that [¹²⁵I]-16α-iodo-3,17β-E2 had similar binding affinity for the mER receptor as it did for the ERα receptor (118 pM vs. 124 pM). However, the total number of mER binding sites was about 5-fold lower than the ERα membrane estrogen receptor polypeptide membrane estrogen receptor polypeptide membrane estrogen receptor polypeptide receptor (32 fmol/mg protein vs. 155 fmol/mg protein).

The present invention also contemplates an assay method and system for identifying selective mER receptor ligands. The method involves detecting binding of a test compound to cells containing the mER receptor. The assay system comprises cells that express mER receptors, where the number of cells in the assay system is sufficient to detect an alteration in calcium mobilization. The test system also includes an appropriate cell culture medium to permit cell growth and viability, and preferably tissue culture plates or arrays containing the host cells in cell culture medium.

The invention also discloses a method for identifying a test compound that antagonizes or agonizes mER receptors. The method comprises detecting an increase (agonist) or decrease of an agonist induced increase (antagonist) in calcium mobilization in the assay system when contacted with the test compound.

Thus, the present invention advantageously provides mER protein, including fragments, derivatives, and analogs of mER; mER nucleic acids, including oligonucleotide primers and probes, and mER regulatory sequences (especially an mER primer and splice sites with introns); mER-specific antibodies; and related methods of using these materials to detect the presence of mER proteins or nucleic acids, mER binding partners, and in screens for agonists and antagonists of mER.

General Definitions

As used herein, the term "isolated" means that the referenced material is removed from the environment in which it is normally found. Thus, an isolated biological material can be free of cellular components, i.e., components of the cells in which the material is found or produced in nature. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. A protein expressed from a vector in a cell, particularly a cell in which the protein is normally not expressed is also a regarded as isolated. An isolated organelle, cell, or tissue is removed from the anatomical site in which it is found in a cell or an organism. An isolated material may be, but need not be, purified.

The term "purified" as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term "substantially free" is used operationally, in the context of analytical testing of the material. Preferably, purified material substantially free of contaminants is at least 50% pure; more preferably, at least 90% pure; and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The "substantially pure" indicates the highest degree of purity which can be achieved using conventional purification techniques known in the art.

In a specific embodiment, the term "about" or "approximately" means within a scientifically acceptable error range for a given value relative to the precision with which the value is or can be measured, e.g., within 20%, preferably within 10%, and more preferably within 5% of a given value or range. Alternatively, particularly with biological systems, the term can mean within an order of magnitude, preferably within 5-fold and more preferably within 2-fold of a given value.

A "sample" as used herein refers to a biological material which can be tested for the presence of mER protein or mER nucleic acids. Such samples can be obtained from cell lines and animal subjects, such as humans and non-human animals, and include tissue, especially muscle, biopsies, blood and blood products; plural effusions; cerebrospinal fluid (CSF); ascites fluid; and cell culture.

Non-human animals include, without limitation, laboratory animals such as mice, rats, rabbits, hamsters, guinea pigs, etc.; domestic animals such as dogs and cats; and, farm animals such as sheep, goats, pigs, horses, and cows.

The use of italics indicates a nucleic acid molecule; normal text indicates the polypeptide or protein.

The term Aligand@ refers to a compound that recognizes and binds to a receptor binding site. In a specific embodiment, the ligand binds to the mER receptors of the invention. Upon binding to the receptor, the ligand may produce agonist or antagonist functional effects. Ligands may be radiolabeled in order to localize receptor expression and assess receptor binding.

The term Aagonist@ refers to a ligand that binds to the receptor and produces a functional effect similar to that produced by the endogenous ligand for the receptor. As used herein, an agonist encompasses full agonists (ligands that produce the same maximal effect as the endogenous ligand) and partial agonists (ligands that produce less than the maximal effect produced by the endogenous ligand). In a specific embodiment, the agonist at the mER receptor produces an effect similar to that produced by estrogen, the proposed endogenous ligand for the mER receptor. Examples of such agonists include, but are not limited to, 16α-iodo-E2, E2, raloxifone, and estrone.

The term Aantagonist@ refers to a ligand that binds to the receptor and blocks a functional effect produced by an agonist for the receptor or the endogenous ligand of the receptor. Examples of such antagonists include, but are not limited to, ICI-182780.

The phrase Acompound selective@ or Acompound selectivity@ refers to the ability of a mER agonist or antagonist to elicit a response from the mER receptor while eliciting minimal responses from another receptor. Stated differently, a selective mER agonist may be a potent agonist for the mER receptor while agonizing another receptor, such as another ER receptor (e.g., ERα), poorly or not at all.

The phrase Areceptor selective@ or Areceptor selectivity@ refers to the a receptor that discriminates between classes of compounds. In other words, a compound may recognize and bind one class of compounds (e.g., steroid) and not another class of compounds (e.g., peptides). In one embodiment, the mER of the present invention is selective for steroids, more preferably estrogen. In an additional embodiment, the mER is selective for estrogen versus other steroids.

The term Aability to elicit a response@ refers to the ability of a mER agonist or antagonist ligand to agonize or antagonize mER receptor activity, respectively.

As used herein the term "transformed cell" refers to a modified host cell that expresses a functional mER receptor expressed from a vector encoding the estrogen receptor. Any cell can be used.

A "functional estrogen receptor" is a receptor that binds estrogen or mER agonists and transduces a signal upon such binding. Preferably, the signal that is transduced is calcium mobilization however, other signaling pathways may be activated by mER. For example, phosphorylation of kinases and various other proteins involved in signal transduction. The mER receptors may be derived from a variety of sources, including mammal, e.g., human, bovine, mouse, primate, porcine, canine, and rat; and avian. The receptor also may be derived from immortalized cell lines such as, but not limited to, neuronal (SY5Y, HT22, D12, H19-7), breast cancer cell lines BFN28, (MCF7), ovarian (primary rat granulosa), endothelial (D12) and pancreatic (RINm5F).

The cells of the invention are particularly suitable for an assay system for mER receptor ligands that modulate second messenger levels. An "assay system" is one or more collections of such cells, e.g., in a microwell plate or some other culture system. To permit evaluation of the effects of a test compound on the cells, the number of cells in a single assay system is sufficient to express a detectable amount of the regulated second messenger at least under conditions of maximum second messenger formation and/or accumulation.

A Asecond messenger@ is an intracellular molecule or ion, where formation and/or accumulation of the second messenger is regulated by activation of cellular membranes. In one embodiment, cellular membranes contain G-protein coupled receptor, ion channels, and tyrosine kinase receptors. In the context of this invention, the cellular membrane contains a mER receptor as defined herein. In a specific embodiment, the second messenger is one or more of cAMP, cGMP, inositol phosphate, diacyl glycerol, and ions such as calcium and potassium. Preferably, the second messenger is calcium.

A "test compound" or Acandidate compound@ is any molecule that can be tested for its ability to bind mER receptors, and preferably modulate second messenger accumulation through the mER receptor, as set forth herein. A compound that binds, and preferably modulates mER is a Alead compound@ suitable for further testing and development as an mER agonist or antagonist. As used herein, the term Aprovide@ refers to supplying the compounds or pharmaceutical compositions of the present invention to cells or to an animal, preferably a human, in any form. For example, a prodrug form of the compounds may be provided the subject, which then is metabolized to the compound in the body.

The term AP2" or AP2 fraction@, as used herein, refers to the pellet obtained from centrifugation of a cell culture or tissue that is homogenized. Typically, the homogenate is then centrifuged. The resulting pellet and supernatant are termed P1 and S1, respectively. The S1 is then centrifuged to produce a second pellet and supernatant termed P2 and S2, respectively. Herein, the P2 contains enriched subcellular components such as the plasma membrane whereas the S2 contains soluble intracellular molecules (cytosol).

mER Receptor

The mER receptor, as defined herein, refers to a polypeptide present in the P2 cellular fraction. Additionally, ERα specific antibodies have affinity for the polypeptide. Additionally, the protein has an apparent molecular weight of about 67 kDa, based on SDS-PAGE. Activation of the receptor regulates calcium mobilization.

The P2 fraction can be prepared by any methods known in the art such as, but not limited to, centrifugation separation. In one embodiment, the tissue source or cells are mechanically disrupted (e.g., homogenization or sonication). The tissue or cells are then centrifuged to remove extracellular debris and intact cells. Typically, this centrifugation is performed at a low speed (e.g., 5,000 to 20,000 x g) and ice-cold temperatures to pellet out the heavier components. However, any speed and temperature defined by one of ordinary skill in the art may be used. The supernatant obtained from the centrifugation is then centrifuged to separate cytosolic components from the particulate components. Typically, this centrifugation is performed at a higher speed (e.g., 100,000 x g) and ice-cold temperatures. Again, any speed and temperature defined by one of ordinary skill in the art may be used. The length of the centrifugation cycles also may be determined and optimized by one of ordinary skill in the art. In one specific embodiment, the P2 cellular fraction is prepared by homogonization of the cells and centrifugation at 15,000 x g for 15 min at 4ΕC. The resulting supernatant then is homogenized and membranes were isolated by centrifugation at 100,000 x g for 1h at 4ΕC. The particulate fraction (observed as a pellet in the centrifugation tube) obtained following the spin is labeled P2 (membranes) and contains the mER.

Antibody studies indicate that the mER protein has significant homology to the known nuclear ERα receptor. Western blots with numerous antibodies indicates cross-reactivity of the antibody between the mER protein and the nuclear ER receptor. Lack of cross-reactivity by specific antibodies, 3E6-F2, 16D4-G2, 8A11-F6, SRA1000, 7A9-E1, and 2D4-F5 indicates that the epitopes recognized by these antibodies are different between the mER and nuclear ERα (Table 2).

Radioligand binding studies indicate the presence of a saturable high affinity binding site in membrane preparations from rat neuronal tissue. Specifically, studies showed saturable binding in membrane preparations from the anterior pituitary, hippocampus, and hypothalamus. These studies indicate the presence of a saturable membrane-associated ER site, similar to that defined in the present application. These localization studies suggest that a similar protein may play a role in hormone secretion.

Screening of cell lines indicated that the mER protein was present in the SY5Y, HT22, D12, MCF7, rat granulosa, and RINm5F cell lines. Additionally, the nuclear ERα protein also was detected in the D12, MCF7, rat granuola, HT22, and RINm5F cell lines. One cell line screened from a neuroblastoma line, SHEP, only showed binding for nuclear ER.

The molecular weight of the protein of the present invention may be assessed by any method known in the art such as, but not limited to, mass spectrometry, gel-filtration chromatography, and SDS polyacrylamide gel electrophoresis (SDS-PAGE). Preferably, SDS-PAGE is used. Methods of SDS-PAGE are known in the art (Sambrook, infra.)

Ligand interaction with mER receptors modulates calcium mobilization and may be used to modulate/regulate cell cycle and cell cycle functions. Modulation of mER receptors may be a treatment for disease states such as, but not limited to, neurodegeneration, cardiovascular disease, infertility, and osteoporosis.

The mER fragments, derivatives, and analogs can be characterized by one or more of the characteristics of mER protein. In a specific embodiment, in order to develop the specific C-terminal and N-terminal mER antibodies, antibodies can be raised against either portion of the mER protein, or antigenic peptides identified using a hydrophobicity profile or other algorithms.

Analogs and derivatives of the mER receptor of the invention have the same or homologous characteristics of mER as set forth above. For example, a truncated form of mER can be provided. Such a truncated form includes mER with a either an N-terminal, C-terminal, or internal deletion. In a specific embodiment, the derivative is functionally active, i.e., capable of exhibiting one or more functional activities associated with a full-length, wild-type mER of the invention. Such functions include, but are not limited to, modulation of calcium mobilization. Alternatively, a mER chimeric fusion protein can be prepared in which the mER portion of the fusion protein has one or more characteristics of mER. Such fusion proteins include fusions of the mER receptor with a marker polypeptide, such as FLAG, a histidine tag, a myc tag, or glutathione-S-transferase (GST). Alternatively, the mER receptor can be fused with an expression-related peptide, such as yeast α-mating factor, a heterogeneous signal peptide, or a peptide that renders the protein more stable upon expression. The mER can also be fused with a unique phosphorylation site for labeling.

Cloning and Expression of mER

The present invention contemplates analysis and isolation of a gene encoding a functional or mutant mER, including a full length, or naturally occurring form of mER, and any antigenic fragments thereof from any source, preferably human. It further contemplates expression of functional or mutant mER protein for evaluation, diagnosis, or therapy.

One of ordinary skill in the art can determine the amino acid and nucleic acid sequences of the present invention using methods well known in the art. For example, a P2 fraction can be obtained from any cell line or tissue source. In one non-limiting protocol, whole cells are homogenized and centrifuged at 15,000 x g for 15 min at 4ΕC. The resulting supernatant then is homogenized and membranes are isolated by centrifugation at 100,000 x g for 1h at 4ΕC. The pellet obtained following the spin is labeled P2. Cells that may be used to determine the sequence include, but are not limited to, SY5Y, HT22, D12, BFN28, MCF7, rat granulosa, and RINm5F cell lines. Preferably, the cell line is D12.

The protein of the present invention can be isolated from the membrane by any method known in the art, such as chromatography (e.g., ion exchange, affinity, immunoaffinity, sizing column, metal-chelate affinity, and high performance liquid), centrifugation, differential solubility, immunoprecipitation, or by any other standard technique used for the purification of proteins.

After isolation the amino acid sequence of the protein can be determined by well established methods and apparatuses that are used in the art today. Such methods include, but are not limited to, 2-D PAGE, mass spectrometry, and Edman degradation. In Edman degradation, a protein's amino-terminal amino acid is specifically reacted with phenylisothiocyanate (PITC). This derivatized amino acid is then selectively removed, leaving the rest of the peptide chain intact. Each cycle of the degradation removes an amino acid from the amino terminal end of the protein or peptide sample. This cyclic process provides the primary structure.

The protein of the present invention is Atranslated@ from a nucleic acid sequence. Thus, mER refers to orthologs and allelic variants, e.g., a protein having greater than about 50%, preferably greater than 80%, more preferably still greater than 90%, and even more preferably greater than 95% overall sequence identity to the present invention. Allelic variants may differ from 1 to about 5 amino residues from the present invention.

An "amino acid sequence" is any chain of two or more amino acids. Each amino acid is represented in DNA or RNA by one or more triplets of nucleotides (see definition infra.). Each triplet forms a Acodon@, corresponding to an amino acid. The genetic code has some redundancy, also called degeneracy, meaning that most amino acids have more than one corresponding codon. For example, the amino acid lysine (Lys) can be coded by the nucleotide triplet or codon AAA or by the codon AAG. Because the nucleotides in DNA and RNA sequences are read in groups of three for protein production, it is important to begin reading the sequence at the correct amino acid, so that the correct triplets are read. The way that a nucleotide sequence is grouped into codons is called the "reading frame."

It is understood by one of ordinary skill in the art that the nucleic acid or nucleotide sequence of the protein of the present invention can be determined from the amino acid sequence. A skilled artisan could use the known amino acid sequence of the protein to produce all the nucleotide sequence combinations that may be translated into the protein of the present invention base don the genetic code and degeneracy that is present. However, it is understood by one of ordinary skill in the art that there are numerous nucleotide sequences that may be determined based on the amino acid sequence. To determine the genomic sequence that encodes the protein of the present invention, Aoligonucleotides@ or Aprobes@, based on proposed nucleic acid sequences may be produced.

As used herein, the term "oligonucleotide" or Aprobe@ refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid (such as in a DNA library). In another embodiment, oligonucleotides (one or both of which may be labeled) can be used as PCR primers, either for cloning full length or a fragment of mER, or to detect the presence of nucleic acids encoding mER. In a further embodiment, an oligonucleotide of the invention can form a triple helix with a mER DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Hybridization of the oligonucleotide to a nucleic acid sequence in a DNA library would indicate the presence of the sequence that encodes the protein of the present invention. The nucleotide can be isolated and the nucleotide sequence can be determined by any method known in the art. Such methods include, but are not limited to, the Sanger method and the Maxam-Gilbert method. Identification of the coding sequence of the protein of the present invention allows one to assess the effect of mutations in the sequence on the function of the protein.

The mER analogs can be made by altering encoding nucleic acid sequences by substitutions, additions or deletions that provide for functionally similar molecules, i.e., molecules that perform one or more mER functions. In a specific embodiment, an analog of mER is a sequence-conservative variant of mER. In another embodiment, an analog of mER is a function-conservative variant. In yet another embodiment, an analog of mER is an allelic variant or a homologous variant from another species. In an embodiment, human variants of mER are described.

The mER derivatives include, but are by no means limited to, phosphorylated mER, glycosylated mER, methylated mER, acylated mER, and other mER proteins that are otherwise chemically modified. The mER derivatives also include labeled variants, e.g., radio-labeled with iodine (or, as pointed out above, phosphorous); a detectable molecule, such as but by no means limited to biotin, a chelating group complexed with a metal ion, a chromophore or fluorophore, a gold colloid, or a particle such as a latex bead; or attached to a water soluble polymer.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein "Sambrook et al., 1989"); DNA Cloning: A Practical Approach, Volumes I and II (D.N. Glover ed. 1985); Oligonucleotide Synthesis (M.J. Gait ed. 1984); Nucleic Acid Hybridization [B.D. Hames & S.J. Higgins eds. (1985)]; Transcription And Translation [B.D. Hames & S.J. Higgins, eds. (1984)]; Animal Cell Culture [R.I. Freshney, ed. (1986)]; Immobilized Cells And Enzymes [IRL Press, (1986)]; B.Perbal, A Practical Guide To Molecular Cloning (1984); F.M. Ausubel et al. (eds.), Current Protocols in Molecular Biology, John Wiley & Sons, Inc. (1994).

Molecular Biology - Definitions

"Amplification" of DNA as used herein denotes the use of polymerase chain reaction (PCR) to increase the concentration of a particular DNA sequence within a mixture of DNA sequences. For a description of PCR see Saiki et al., Science, 239:487, 1988.

"Chemical sequencing" of DNA denotes methods such as that of Maxam and Gilbert (Maxam-Gilbert sequencing, Maxam and Gilbert, Proc. Natl. Acad. Sci. USA 1977, 74:560), in which DNA is randomly cleaved using individual base-specific reactions.

"Enzymatic sequencing" of DNA denotes methods such as that of Sanger (Sanger et al., Proc. Natl. Acad. Sci. USA 1977, 74:5463, 1977), in which a single-stranded DNA is copied and randomly terminated using DNA polymerase, including variations thereof well-known in the art.

As used herein, "sequence-specific oligonucleotides" refers to related sets of oligonucleotides that can be used to detect allelic variations or mutations in the mER gene.

A "nucleic acid molecule" refers to the phosphate ester polymeric form of ribonucleosides (adenosine, guanosine, uridine or cytidine; "RNA molecules") or deoxyribonucleosides (deoxyadenosine, deoxyguanosine, deoxythymidine, or deoxycytidine; "DNA molecules"), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5' to 3' direction along the nontranscribed strand of DNA (i.e., the strand having a sequence homologous to the mRNA). A "recombinant DNA molecule" is a DNA molecule that has undergone a molecular biological manipulation.

A "polynucleotide" or "nucleotide sequence" is a series of nucleotide bases (also called "nucleotides") in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as "protein nucleic acids" (PNA) formed by conjugating bases to an amino acid backbone. This also includes nucleic acids containing modified bases, for example thio-uracil, thio-guanine and fluoro-uracil.

The nucleic acid molecules (polynucleotides) herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5'- and 3'- non-coding regions, and the like. The nucleic acids may also be modified by many means known in the art. Non-limiting examples of such modifications include methylation, "caps", substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A "coding sequence" or a sequence "encoding" an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term "polypeptide" refers to a polymer of amino acids and does not refer to a specific length of the product; thus, peptides, oligopeptides, and proteins are included within the definition of polypeptide. This term also does not refer to, or exclude, post translational modifications of the polypeptide, for example, glycosylations, acetylations, phosphorylations, and the like.

The term "gene", also called a "structural gene" means a DNA sequence that codes for or corresponds to a particular sequence of amino acids which comprise all or part of one or more proteins or enzymes, and may or may not include introns and regulatory DNA sequences, such as promoter sequences, 5'-untranslated region, or 3'-untranslated region which affect for example the conditions under which the gene is expressed.

A "promoter sequence" is a DNA regulatory region capable of binding RNA polymerase in a cell and initiating transcription of a downstream (3' direction) coding sequence. For purposes of defining the present invention, the promoter sequence is bounded at its 3' terminus by the transcription initiation site and extends upstream (5' direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The present invention includes the mER receptor gene promoter found in the genome, which can be operatively associated with a mER coding sequence with a heterologous coding sequence.

Promoters which may be used to control gene expression include, but are not limited to, cytomegalovirus (CMV) promoter (U.S. Patents No. 5,385,839 and No. 5,168,062), the SV40 early promoter region (Benoist and Chambon, Nature 1981, 290:304-310), the promoter contained in the 3' long terminal repeat of Rous sarcoma virus (Yamamoto, et al., Cell 1980, 22:787-797), the herpes thymidine kinase promoter (Wagner et al., Proc. Natl. Acad. Sci. USA 1981, 78:1441-1445), the regulatory sequences of the metallothionein gene (Brinster et al., Nature 1982, 296:39-42); prokaryotic expression vectors such as the beta-lactamase promoter (Villa-Komaroff, et al., Proc. Natl. Acad. Sci. USA 1978, 75:3727-3731), or the tac promoter (DeBoer, et al., Proc. Natl. Acad. Sci. USA 1983, 80:21-25); see also "Useful proteins from recombinant bacteria" in Scientific American 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985, 315:338-340; Kollias et al., Cell 1986, 46:89-94), hematopoietic stem cell differentiation factor promoters, erythropoietin receptor promoter (Maouche et al., Blood 1991, 15:2557), etc.

The term "host cell" means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

A coding sequence is "under the control of" or "operatively associated with@ transcriptional and translational control sequences in a cell when RNA polymerase transcribes the coding sequence into mRNA, which is then trans-RNA spliced (if it contains introns) and translated, in the case of mRNA, into the protein encoded by the coding sequence.

The terms "express" and "expression" mean allowing or causing the information in a gene or DNA sequence to become manifest, for example producing a protein by activating the cellular functions involved in transcription and translation of a corresponding gene or DNA sequence. A DNA sequence is expressed in or by a cell to form an Aexpression product@ such as a protein. The expression product itself , e.g., the resulting protein, may also be said to be Aexpressed@ by the cell. An expression product can be characterized as associated with the plasma membrane. A substance is Aassociated with the plasma membrane@ if it interacts in significant measure with the membrane. A substance is Asecreted@ by a cell if it appears in significant measure outside the cell, from somewhere on or inside the cell.

The terms Atransformation@ and Atransfection@ mean the introduction of a Aforeign@ (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a Acloned@ or Aforeign@ gene or sequence, may include regulatory or control sequences, such as start, stop, promoter, signal, secretion, or other sequences used by a cell=s genetic machinery. The gene or sequence may include nonfunctional sequences or sequences with no known function. A host cell that receives and expresses introduced DNA or RNA has been Atransformed@ and is a "transformant" or a "clone." The DNA or RNA introduced to a host cell can come from any source, including cells of the same genus or species as the host cell, or cells of a different genus or species.

The terms "vector", "cloning vector" and "expression vector" mean the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A "cassette" refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a ADNA construct.@ A common type of vector is a Aplasmid@, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clontech), pUC plasmids, pET plasmids (Novagen, Inc., Madison, WI), pRSET or pREP plasmids (Invitrogen, San Diego, CA), or pMAL plasmids (New England BioLabs, Beverly, MA), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

The term "expression system" means a host cell and compatible vector under suitable conditions, e.g., for the expression of a protein coded for by foreign DNA carried by the vector and introduced to the host cell. Common expression systems include E. coli host cells and plasmid vectors, insect host cells and Baculovirus vectors, and mammalian host cells and vectors.

The term "heterologous" refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature. In the context of the present invention, an mER gene is heterologous to the vector DNA in which it is inserted for cloning or expression, and it is heterologous to a host cell containing such a vector, in which it is expressed.

The terms "mutant" and "mutation" mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. The term Avariant@ may also be used to indicate a modified or altered gene, DNA sequence, enzyme, cell, etc., i.e., any kind of mutant.

"Sequence-conservative variants" of a polynucleotide sequence are those in which a change of one or more nucleotides in a given codon position results in no alteration in the amino acid encoded at that position.

"Function-conservative variants" are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A "function-conservative variant" also includes a polypeptide or enzyme which has at least 60 % amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

As used herein, the term "homologous" in all its grammatical forms and spelling variations refers to the relationship between proteins that possess a "common evolutionary origin," including proteins from superfamilies (e.g., the immunoglobulin superfamily) and homologous proteins from different species (e.g., myosin light chain, etc.) (Reeck et al., Cell 1987, 50:667). Such proteins (and their encoding genes) have sequence homology, as reflected by their sequence similarity, whether in terms of percent similarity or the presence of specific residues or motifs at conserved positions.

Accordingly, the term "sequence similarity" in all its grammatical forms refers to the degree of identity or correspondence between nucleic acid or amino acid sequences of proteins that may or may not share a common evolutionary origin (see Reeck et al., supra). However, in common usage and in the instant application, the term "homologous," when modified with an adverb such as "highly," may refer to sequence similarity and may or may not relate to a common evolutionary origin.

In a specific embodiment, two DNA sequences are "substantially homologous" or "substantially similar" when at least about 80%, and most preferably at least about 90 or 95% of the nucleotides match over the defined length of the DNA sequences, as determined by sequence comparison algorithms, such as BLAST, FASTA, DNA Strider, etc. An example of such a sequence is an allelic or species variant of the specific mER gene of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are "substantially homologous" or "substantially similar" when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wisconsin) pileup program, or any of the programs described above (BLAST, FASTA, etc)

A nucleic acid molecule is "hybridizable" to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the "stringency" of the hybridization. For preliminary screening for homologous nucleic acids, low stringency hybridization conditions, corresponding to a Tm (melting temperature) of 55ΕC, can be used, e.g., 5x SSC, 0.1% SDS, 0.25% milk, and no formamide; or 30% formamide, 5x SSC, 0.5% SDS. Moderate stringency hybridization conditions correspond to a higher Tm, e.g., 40% formamide, with 5x or 6x SSC. High stringency hybridization conditions correspond to the highest Tm, e.g., 50% formamide, 5x or 6x SSC. SSC is a 0.15M NaCl, 0.015M Na-citrate buffer. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term "standard hybridization conditions" refers to a Tm of 55ΕC, and utilizes conditions as set forth above. In a preferred embodiment, the Tm is 60ΕC; in a more preferred embodiment, the Tm is 65ΕC. In a specific embodiment, Ahigh stringency@ refers to hybridization and/or washing conditions at 68ΕC in 0.2xSSC, at 42ΕC in 50% formamide, 4xSSC, or under conditions that afford levels of hybridization equivalent to those observed under either of these two conditions.

The present invention provides antisense nucleic acids (including ribozymes), which may be used to inhibit expression of mER of the invention. Inhibition of mER expression may be desired when upregulation of mER receptor expression or inhibition of mER induced modulation of calcium mobilization is needed. An "antisense nucleic acid" is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, "antisense" broadly includes RNA-RNA interactions, RNA-DNA interactions, ribozymes and RNase-H mediated arrest. Antisense nucleic acid molecules can be encoded by a recombinant gene for expression in a cell (e.g., U.S. Patent No. 5,814,500; U.S. Patent No. 5,811,234), or alternatively they can be prepared synthetically (e.g., U.S. Patent No. 5,780,607).

Specific non-limiting examples of synthetic oligonucleotides envisioned for this invention include oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂-NH-O-CH₂, CH₂-N(CH₃)-O-CH₂, CH₂-O-N(CH₃)-CH₂, CH₂-N(CH₃)-N(CH₃)-CH₂ and O-N(CH₃)-CH₂-CH₂ backbones (where phosphodiester is O-PO₂-O-CH₂). U.S. Patent No. 5,677,437 describes heteroaromatic olignucleoside linkages. Nitrogen linkers or groups containing nitrogen can also be used to prepare oligonucleotide mimics (U.S. Patents No. 5,792,844 and No. 5,783,682). U.S. Patent No. 5,637,684 describes phosphoramidate and phosphorothioamidate oligomeric compounds. Also envisioned are oligonucleotides having morpholino backbone structures (U.S. Patent No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2' position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-; S-, or N-alkyl; O-, S-, or N-alkenyl; SOCH₃; SO2CH₃; ONO₂; NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

mER Nucleic Acids

A gene encoding mER, whether genomic DNA or cDNA, can be isolated from any source, particularly from a human cDNA or genomic library. Methods for obtaining mER gene are well known in the art, as described above (see, e.g., Sambrook et al., 1989, supra). The DNA may be obtained by standard procedures known in the art from cloned DNA (e.g., a DNA "library"), and preferably is obtained from a cDNA library prepared from tissues with high level expression of the protein, by chemical synthesis, by cDNA cloning, or by the cloning of genomic DNA, or fragments thereof, purified from the desired cell (See, for example, Sambrook et al., 1989, supra; Glover, D.M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene. Identification of the specific DNA fragment containing the desired mER gene may be accomplished in a number of ways. For example, a portion of a mER gene exemplified infra can be purified and labeled to prepare a labeled probe, and the generated DNA may be screened by nucleic acid hybridization to the labeled probe (Benton and Davis, Science 1977, 196:180; Grunstein and Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous mER gene.

Further selection can be carried out on the basis of the properties of the gene, e.g., if the gene encodes a protein product having the isoelectric, electrophoretic, amino acid composition, partial or complete amino acid sequence, antibody binding activity, or ligand binding profile of mER protein as disclosed herein. Thus, the presence of the gene may be detected by assays based on the physical, chemical, immunological, or functional properties of its expressed product.

Other DNA sequences which encode substantially the same amino acid sequence as a mER gene may be used in the practice of the present invention. These include but are not limited to allelic variants, species variants, sequence conservative variants, and functional variants.

Amino acid substitutions may also be introduced to substitute an amino acid with a particularly preferable property. For example, a Cys may be introduced a potential site for disulfide bridges with another Cys.

The genes encoding mER derivatives and analogs of the invention can be produced by various methods known in the art. The manipulations which result in their production can occur at the gene or protein level. For example, the cloned mER gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of mER, care should be taken to ensure that the modified gene remains within the same translational reading frame as the mER gene, uninterrupted by translational stop signals, in the gene region where the desired activity is encoded.

Additionally, the nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Such modifications can be made to introduce restriction sites and facilitate cloning the mER gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB¨ linkers (Pharmacia), etc. PCR techniques are preferred for site directed mutagenesis (see Higuchi, 1989, "Using PCR to Engineer DNA", in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as Bluescript, pBR322 derivatives or pUC plasmid derivatives, e.g., pGEX vectors, pMal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In addition, simple PCR or overlapping PCR may be used to insert a fragment into a cloning vector.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2Φ plasmid.

mER Regulatory Nucleic Acids

Elements of the mER promoter can be identified by scanning the genomic region upstream of the mER start site, e.g., by creating deletion mutants and checking for expression, or with the TRANSFAC algorithm. Sequences up to about 6 kilobases (kb) or more upstream from the mER start site can contain tissue-specific regulatory elements.

The term "mER promoter" encompasses artificial promoters. Such promoters can be prepared by deleting nonessential intervening sequences from the upstream region of the mER promoter, or by joining upstream regulatory elements from the mER promoter with a heterologous minimal promoter, such as the CMV immediate early promoter.

An mER promoter can be operably associated with a heterogenous coding sequence, e.g., for reporter gene (luciferase and green fluorescent proteins are examples of reporter genes) in a construct. This construct will result in expression of the heterologous coding sequence under control the mER promoter, e.g., a reporter gene can be expressed, under conditions that under normal conditions cause mER expression. This construct can be used in screening assays, described below, for mER agonists and antagonists.

Expression of mER Polypeptides

The nucleotide sequence coding for mER, or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Thus, a nucleic acid encoding mER of the invention can be operationally associated with a promoter in an expression vector of the invention. Both cDNA and genomic sequences can be cloned and expressed under control of such regulatory sequences. Such vectors can be used to express functional or functionally inactivated mER polypeptides.

The necessary transcriptional and translational signals can be provided on a recombinant expression vector, or they may be supplied by the native gene encoding mER and/or its flanking regions.

Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of mER protein may be controlled by any promoter/enhancer element known in the art, but these regulatory elements must be functional in the host selected for expression. Promoters which may be used to control mER gene expression include, but are not limited to, cytomegalovirus (CMV) promoter (U.S. Patent Nos. 5,385,839 and 5,168,062), the SV40 early promoter region (Benoist and Chambon, 1981, Nature 290:304-310), the promoter contained in the 3' long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982), prokaryotic expression vectors such as the β-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983; see also "Useful proteins from recombinant bacteria" in Scientific American, 242:74-94, 1980), promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit tissue specificity, particularly endothelial cell-specific promoters.

Solubilized forms of the protein can be obtained by solubilizing inclusion bodies or reconstituting membrane components, e.g., by treatment with detergent, and if desired sonication or other mechanical processes, as described above. The solubilized protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Vectors

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX (Smith et al., Gene 67:31-40, 1988), pMB9 and their derivatives, plasmids such as RP4; phage DNAS, e.g., the numerous derivatives of phage l, e.g., NM989, and other phage DNA, e.g., M13 and filamentous single stranded phage DNA; yeast plasmids such as the 2Φ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Viral vectors, such as lentiviruses, retroviruses, herpes viruses, adenoviruses, adeno-associated viruses, vaccinia virus, baculovirus, alphavirus, and other recombinant viruses with desirable cellular tropism are also useful. Thus, a gene encoding a functional or mutant mER protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part) or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 2:320-330, 1991), defective herpes virus vector lacking a glyco-protein L gene (Patent Publication RD 371005 A), or other defective herpes virus vectors (International Patent Publication No. WO 94/21807, published September 29, 1994; International Patent Publication No. WO 92/05263, published April 2, 1994); an attenuated adenovirus vector, such as the vector described by Stratford-Perricaudet et al. (J. Clin. Invest. 90:626-630, 1992; see also La Salle et al., Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, CA; AAV vectors), Cell Genesys (Foster City, CA; retroviral, adenoviral, AAV vectors, and lentiviral vectors), Clontech (retroviral and baculoviral vectors), Genovo, Inc. (Sharon Hill, PA; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be provided to block humoral or cellular immune responses to the viral vectors (see, e.g., Wilson, Nature Medicine, 1995). In that regard, it is advantageous to employ a viral vector that is engineered to express a minimal number of antigens.

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer, et al., Science 259:1745-1748, 1993). Useful lipid compounds and compositions for transfer of nucleic acids are described in International Patent Publications WO 95/18863 and WO 96/17823, and in U.S. Patent No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO 95/21931), peptides derived from DNA binding proteins (e.g., International Patent Publication WO 96/25508), or a cationic polymer (e.g., International Patent Publication WO95/21931).

Alternatively, non-viral DNA vectors for gene therapy can be introduced into the desired host cells by methods known in the art, e.g., electroporation, microinjection, cell fusion, DEAE dextran, calcium phosphate precipitation, use of a gene gun (ballistic transfection; see, e.g., U.S. Pat. No. 5,204,253, U.S. Pat. No. 5,853,663, U.S. Pat. No. 5,885,795, and U.S. Pat. No. 5,702,384 and see Sanford, TIB-TECH, 6:299-302, 1988; Fynan et al., Proc. Natl. Acad. Sci. U.S.A., 90:11478-11482, 1993; and Yang et al., Proc. Natl. Acad. Sci. U.S.A., 87:1568-9572, 1990), or use of a DNA vector transporter (see, e.g., Wu, et al., J. Biol. Chem. 267:963-967, 1992; Wu and Wu, J. Biol. Chem. 263:14621-14624, 1988; Hartmut, et al., Canadian Patent Application No. 2,012,311, filed March 15, 1990; Williams, et al., Proc. Natl. Acad. Sci. USA 88:2726-2730, 1991). Receptor-mediated DNA delivery approaches can also be used (Curiel, et al., Hum. Gene Ther. 3:147-154, 1992; Wu and Wu, J. Biol. Chem. 262:4429-4432, 1987). U.S. Patent Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir, et al., C.P. Acad. Sci., 321:893, 1998; WO 99/01157; WO 99/01158; WO 99/01175).

mER Ligands and Binding Partners

The present invention further permits identification of physiological ligands and binding partners of mER. One method for evaluating and identifying mER binding partners is the yeast two-hybrid screen. Preferably, the yeast two-hybrid screen is performed using an cell library with yeast that are transformed with recombinant mER. Alternatively, mER can be used as a capture or affinity purification reagent. In another alternative, labeled mER can be used as a probe for binding, e.g., by immunoprecipitation or Western analysis.

Generally, binding interactions between mER and any of its binding partners will be strongest under conditions approximating those found in the cytoplasm, i.e., physiological conditions of ionic strength, pH and temperature. Perturbation of these conditions will tend to disrupt the stability of a binding interaction.

Antibodies to mER

Antibodies to mER are useful, inter alia, for diagnostics and intracellular regulation of mER activity, as set forth below. According to the invention, a mER polypeptide produced recombinantly or by chemical synthesis, and fragments or other derivatives or analogs thereof, including fusion proteins, may be used as immunogens to generate antibodies that recognize the mER polypeptide. Such antibodies include but are not limited to polyclonal, monoclonal, chimeric, single chain, Fab fragments, and an Fab expression library. Such an antibody is preferably specific for human mER and it may recognize either a mutant form of mER or wild-type mER, or both.

One can use the hydropathic index of amino acids, as discussed by Kate and Doolittle (J Mol Biol. 1982, 157:105-132). See, for example, U.S. Patent 4,554,101, which states that the greatest local average hydrophilicity of a Aprotein,@ as governed by the hydrophilicity of its adjacent amino acids, correlates with its immunogenicity. Accordingly, it is noted that substitutions can be made based on the hydrophilicity assigned to each amino acid. In using either the hydrophilicity index or hydropathic index, which assigns values to each amino acid, it is preferred to introduce substitutions of amino acids where these values are ∀ 2, with ∀ 1 being particularly preferred, and those within ∀ 0.5 being the most preferred substitutions.

Various procedures known in the art may be used for the production of polyclonal antibodies to mER polypeptide or derivative or analog thereof. For the production of antibody, various host animals can be immunized by injection with the mER polypeptide, or a derivative (e.g., fragment or fusion protein) thereof, including but not limited to rabbits, mice, rats, sheep, goats, etc. In one embodiment, the mER polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the mER polypeptide, or fragment, analog, or derivative thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). In fact, according to the invention, techniques developed for the production of "chimeric antibodies" (Morrison et al., J. Bacteriol. 1984, 159:870; Neuberger et al., Nature 1984, 312:604-608; Takeda et al., Nature 1985, 314:452-454) by splicing the genes from a mouse antibody molecule specific for an mER polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

Antibody fragments which contain the idiotype of the antibody molecule can be generated by known techniques. For example, such fragments include but are not limited to: the F(ab')2 fragment which can be produced by pepsin digestion of the antibody molecule; the Fab' fragments which can be generated by reducing the disulfide bridges of the F(ab')2 fragment, and the Fab fragments which can be generated by treating the antibody molecule with papain and a reducing agent.

According to the invention, techniques described for the production of single chain antibodies (U.S. Patent Nos. 5,476,786, 5,132,405, and U.S. Patent 4,946,778) can be adapted to produce mER polypeptide-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for an mER polypeptide, or its derivatives, or analogs.

In the production and use of antibodies, screening for or testing with the desired antibody can be accomplished by techniques known in the art, e.g., radioimmunoassay, ELISA (enzyme-linked immunosorbant assay), "sandwich" immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of an mER polypeptide, one may assay generated hybridomas for a product which binds to an mER polypeptide fragment containing such epitope. For selection of an antibody specific to an mER polypeptide from a particular species of animal, one can select on the basis of positive binding with mER polypeptide expressed by or isolated from cells of that species of animal.

The foregoing antibodies can be used in methods known in the art relating to the localization and activity of the mER polypeptide, e.g., for Western blotting, imaging mER polypeptide in situ, measuring levels thereof in appropriate physiological samples, etc. using any of the detection techniques mentioned above or known in the art. Such antibodies can also be used in assays for ligand binding, e.g., as described in U.S. Patent No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In a specific embodiment, antibodies that act as ligands and agonize or antagonize the activity of mER polypeptide can be generated. In addition, intracellular single chain Fv antibodies can be used to regulate cAMP formation (Marasco et al., Proc. Natl. Acad. Sci. U.S.A. 1993, 90:7884-7893; Chen., Mol. Med. Today 1997, 3:160-167; Spitz et al., Anticancer Res. 1996, 16:3415-22; Indolfi et al., Nat. Med. 1996, 2:634-635; Kijma et al., Pharmacol. Ther. 1995, 68:247-267). Such antibodies can be tested using the assays described infra for identifying ligands.

In another specific embodiment, antibodies can be used to identify the presence of mER protein. In other words, an antibody can be used to localize a protein that comprises the epitopes recognized by the antibody. Upon isolation and purification of the protein, the pharmacological profile of the protein can be determined (e.g., ligand binding profile, molecular weight, agonist activity). If the profile of the isolated protein is similar to the protein of the present invention, it can be determined that the isolated protein is a mER receptor. However, if the profile is different the protein may represent a known ER receptor (such as ERα) or a novel ER receptor subtype. Further pharmacological studies and sequence analysis can be used to define the protein.

Screening and Chemistry

According to the present invention, nucleotide sequences encoding mER is a useful target to identify drugs that are effective in treating disorders associated with estrogen-regulated processes. Drug targets include without limitation (i) isolated nucleic acids derived from the gene encoding mER (e.g., antisense or ribozyme molecules) and (ii) small molecule compounds that recognize and bind the mER receptor.

In particular, identification and isolation of mER provides for development of screening assays, particularly for high throughput screening of molecules that up- or down-regulate the activity of mER. Accordingly, the present invention contemplates methods for identifying specific estrogen receptor ligands that interact with mER receptors, using various screening assays known in the art.

Any screening technique known in the art can be used to screen for mER agonists or antagonists. The present invention contemplates screens for small molecule ligands or ligand analogs and mimics, as well as screens for natural ligands that bind to and agonize or antagonize mER activity in vivo. For example, natural products libraries can be screened using assays of the invention for molecules that agonize or antagonize mER expression or activity.

Another approach uses recombinant bacteriophage to produce large libraries. Using the "phage method" (Scott and Smith, Science 1990, 249:386-390; Cwirla, et al., Proc. Natl. Acad. Sci., USA 1990, 87:6378-6382; Devlin et al., Science 1990, 49:404-406), very large libraries can be constructed (106-108 chemical entities). A second approach uses primarily chemical methods, of which the Geysen method (Geysen et al., Molecular Immunology 1986, 23:709-715; Geysen et al. J. Immunologic Method 1987 102:259-274) and the method of Fodor et al. (Science 1991, 251:767-773) are examples. Furka et al. (14th International Congress of Biochemistry, Volume #5 1988, Abstract FR:013; Furka, Int. J. Peptide Protein Res. 1991, 37:487-493), Houghton (U.S. Patent No. 4,631,211) and Rutter (U.S. Patent No. 5,010,175) describe methods to produce a mixture of peptides that can be tested as agonists or antagonists.

In another aspect, synthetic libraries (Needels et al., Proc. Natl. Acad. Sci. USA 1993, 90:10700-4; Ohlmeyer et al., Proc. Natl. Acad. Sci. USA 1993, 90:10922-10926; Lam et al., PCT Publication No. WO 92/00252; Kocis et al., PCT Publication No. WO 9428028) and the like can be used to screen for ligands that regulate mER activity. Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, NJ), Brandon Associates (Merrimack, NH), and Microsource (New Milford, CT). A rare chemical library is available from Aldrich (Milwaukee, WI). Alternatively, libraries of natural compounds in the form of bacterial, fungal, plant and animal extracts are available from e.g. Pan Laboratories (Bothell, WA) or MycoSearch (NC), or are readily producible. Additionally, natural and synthetically produced libraries and compounds are readily modified through conventional chemical, physical, and biochemical means (Blondelle et al., Tib Tech 1996, 14:60).

Knowledge of the primary sequence of mER, and the similarity of that sequence with proteins of known function, can provide an initial clue as to the structure of agonists or antagonists of the receptor. Identification and screening of agonists antagonists is further facilitated by determining structural features of the receptor, e.g., using X-ray crystallography, neutron diffraction, nuclear magnetic resonance spectrometry, homology studies, structure-activity relationships, and other techniques for structure determination. These techniques provide for the rational design or identification of agonists and antagonists.

One technique that may be used to assess the affinity of a test compound for the mER receptor is a competition binding assay. In this assay, test wells containing an aliquot of a lipid bilayer membranes that contain the estrogen mER receptor are incubated with an known concentration of a radiolabeled ligand for the receptor. The lipid bilayer may be prepared by any known protocol that separates the membrane containing receptor component from the cytosolic components. Each well also is incubated with a different concentration of a unlabeled test compound. Cell membranes are then separated from the incubation mixture by any method known in the art including, but not limited to, centrifugation and vacuum filtration on a cell harvester. The radioactivity of each well is then determined using any device that can detect radioactivity, such as a scintillation counter. As increasing concentrations of the test compound compete for the receptor binding site, the radioactivity detected decreases. The data then can be converted using the Cheng-Prusoff equation (Biochem Pharmacol. 1973, 22:3099-3108) to determine the affinity (Ki) of the compound for the receptor.

Comparison of the affinities of several different ligands at the mER receptor and the nuclear ER receptor allows one to develop a pharmacological profile of the mER receptor. Additionally, these studies may be used to develop a model of the receptor binding site at the mER receptor. Definition of the receptor binding site allows one of ordinary skill in the art to assess positions that increase and decrease binding affinity and activity. In other words, a Apharmacophore@ may be developed. As used herein, a Apharmacophore@ is the minimal three-dimensional orientation of elements needed for receptor binding and/or activity. Comparison of the mER pharmacophore to the nuclear pharmacophore allows the development of ligands that are selective for one receptor versus another.

In vivo screening methods

Intact cells or whole animals expressing a gene encoding mER can be used in screening methods to identify candidate drugs.

In one series of embodiments, a permanent cell line is established. Alternatively, cells (including without limitation mammalian, insect, yeast, or bacterial cells) are transiently programmed to express an mER gene by introduction of appropriate DNA or mRNA. Identification of candidate compounds can be achieved using any suitable assay, including without limitation (i) assays that measure binding of test compounds to mER, (ii) assays that measure the ability of a test compound to modify (i.e., inhibit or enhance) a measurable activity or function of mER, and (iii) assays that measure the ability of a compound to modify (i.e., inhibit or enhance) the transcriptional activity of sequences derived from the promoter (i.e., regulatory) regions of the mER gene.

The mER knockout mammals can be prepared for evaluating the molecular pathology of this defect in greater detail than is possible with human subjects. Such animals also provide excellent models for screening drug candidates. A "knockout mammal" is an mammal (e.g., mouse, rat) that contains within its genome a specific gene that has been inactivated by the method of gene targeting (see, e.g., U.S. Patent Nos. 5,777,195 and 5,616,491). A knockout mammal includes both a heterozygote knockout (i.e., one defective allele and one wild-type allele) and a homozygous mutant (i.e., two defective alleles; a heterologous construct for expression of an mER, such as a human mER, could be inserted to permit the knockout mammal to live if lack of mER expression was lethal). Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou, et al. (Genes and Development 1995, 9:2623-34) describes PPCA knock-out mice.

The term "knockout" refers to partial or complete suppression of the expression of at least a portion of a protein encoded by an endogenous DNA sequence in a cell. The term "knockout construct" refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise (1) a full or partial sequence of one or more exons and/or introns of the gene to be suppressed, (2) a full or partial promoter sequence of the gene to be suppressed, or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases "disruption of the gene" and "gene disruption" refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Generally, the DNA will be at least about 1 kb in length and preferably 3-4 kb in length, thereby providing sufficient complementary sequence for recombination when the knockout construct is introduced into the genomic DNA of the ES cell (discussed below).

Included within the scope of this invention is a mammal in which two or more genes have been knocked out. Such mammals can be generated by repeating the procedures set forth herein for generating each knockout construct, or by breeding to mammals, each with a single gene knocked out, to each other, and screening for those with the double knockout genotype.

Regulated knockout animals can be prepared using various systems, such as the tet-repressor system (see U.S. Patent No. 5,654,168) or the Cre-Lox system (see U.S. Patent Nos. 4,959,317 and 5,801,030).

In another series of embodiments, transgenic animals are created in which (i) a human mER is stably inserted into the genome of the transgenic animal; and/or (ii) the endogenous mER genes are inactivated and replaced with human mER genes. See, e.g., Coffman, Semin. Nephrol. 1997, 17:404; Esther et al., Lab. Invest. 1996, 74:953; Murakami et al., Blood Press. Suppl. 1996, 2:36.

mER Activation Assay

Any cell assay system that allows for assessment of functional activity of mER agonists and antagonists is defined by the present invention. In a specific embodiment, exemplified infra, the assay can be used to identify compounds that selectively interact with mER, which can be evaluated by assessing the effects of cells that express mER and contacted with a test compound, which modulates calcium mobilization. The compounds may be further assessed for effects through known estrogen receptors such as ERα. Compounds that only produce functional effects through the mER receptor are referred to as mER-selective ligand whereas compounds that produce functional effects through another ER receptor than mER receptors are referred to as non-selective ER ligand. The assay system can thus be used to identify compounds that selectively produce a functional effect through estrogen mER receptors. ER-selective ligand are proposed to be compounds that produce non-genomic activities through activation of mER receptors by modulation of calcium mobilization. Compounds that increase calcium mobilization may be useful as novel therapeutics in the prevention of neurodegeneration, cardiovascular disease, infertility, and osteoporosis. Preferably, each experiment is performed in triplicate at multiple different dilutions of test compound.

Alteration in genomic activity refers to changes in gene transcription. In the present invention, alterations in genomic estrogen receptor activity result from an estrogen compound binding to a nuclear estrogen receptor. The estrogen compound/nuclear estrogen receptor complex binds to DNA and activates transcription of genes under the control or regulation of such complexes. The genomic activity of nuclear estrogen receptors generally takes longer to occur than the non-genomic activity of mER. That is, changes in gene transcription due to nuclear estrogen receptor activation take longer to occur than changes, such as nitric oxide production, due to mER activation.

An agonist and/or antagonist screen involves detecting modulation of calcium mobilization by the host cell when contacted with mER ligand. If mobilization is increased, the test compound is a candidate agonist of mER receptors whereas if the agonist induced increase can be blocked by a test compound it is deemed an antagonist for mER. If mobilization is decreased, the test compound is a candidate antagonist of mER receptors.

Any convenient method permits detection of calcium mobilization. For example, calcium flux can be measured by 1-[6-amino-2-(5-carboxy-2-oxazolyl)-5-benzofuranyloxy]- 2-(2-amino-5-methylphenoxy) ethane-N,N,N',N'-tetraacetic acid, pentapotassium salt (FURA-2) fluorescence. FURA-2 A/M complexes calcium present in the system. When whole cells expressing mER are loaded with a fluorescent dye, such as FURA-2, and an estrogen compound is added to these cells, the estrogen compound binds to mER and calcium is released from intracellular stores. The dye chelates these calcium ions and the excitation maximum wavelength of FURA-2 shifts with Ca²⁺ complexation, from 380 nM to 335 nM. Thus, spectrophotometric determination of the ratio for dye:calcium complexes to free dye determines the changes in intracellular calcium concentrations upon addition of an estrogen compound. Many types of instrumentation are now available for FURA-2 experiment. Especially, FURA-2 is suitable for digital imaging microscopy. Other methods that can be used to assess calcium mobilization include, but are not limited to, other Ca²⁺ indicator (fluorescent) dyes, patch clamp technique, addition of radioactive Ca⁺⁴⁵, and pH indicator dyes.

A screen involving alterations in genomic activity such as estrogen-induced progesterone induction (Falkenstein, et al., Pharmacological Reviews 52:513-555, 2000) also may be used. These studies may be used, in addition to binding studies, to assess ligand selectivity. If genomic activity is increased by the compound, the effect may be occurring through a nuclear receptor and the compound is either selective for the nuclear ER receptor or non-selective between the nuclear ER and mER receptors.

The assay system described here also may be used in a high-throughput primary screen for agonists and antagonists, or it may be used as a secondary functional screen for candidate compounds identified by a different primary screen, e.g., a binding assay screen that identifies compounds that interact with the receptor.

High-Throughput Screen

Agents according to the invention may be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time. Such high-throughput screening methods are particularly preferred. The use of high-throughput screening assays to test for agents is greatly facilitated by the availability of large amounts of purified polypeptides, as provided by the invention.

Compounds

An "estrogen compound" is defined as any of the structures described in the 11th edition of "Steroids" from Steraloids Inc., Wilton N. H., here incorporated by reference. Included in this definition are non-steroidal estrogens described in the aforementioned reference. Other estrogen compounds included in this definition are estrogen derivatives, estrogen metabolites, estrogen precursors, selective estrogen receptor modulators (SERMs), and any compound that can bind to an ER. Also included are mixtures of more than one estrogen or estrogen compound. Examples of such mixtures are provided in Table II of U.S. Patent No. 5,554,601 (see column 6). Examples of estrogens having utility either alone or in combination with other agents are provided, e.g., in U.S. Patent No. 5,554,601. In another embodiment, the estrogen compound is a composition of conjugated equine estrogens (PREMARIN^{TM}; Wyeth-Ayerst).

β-estrogen is the β-isomer of estrogen compounds. α-estrogen is the α-isomer of estrogen components. The term "estradiol" is either α- or β-estradiol unless specifically identified.

The term "E2 " is synonymous with β-estradiol, 17β-estradiol, and β-E2. αE2 and α-estradiol is the α isomer of β-E2 estradiol.

In addition, certain compounds, such as the androgen testosterone, can be converted to estradiol in vivo.

Methods of Diagnosis

According to the present invention, genetic variants of mER can be detected to diagnose an mER-associated disease, such as, but not limited to, neurodegeneration, cardiovascular disease, infertility, and osteoporosis. The various methods for detecting such variants are described herein. Where such variants impact mER function, either as a result of a mutated amino acid sequence or because the mutation results in expression of a truncated protein, or no expression at all, they are expected to result in disregulation of calcium mobilization.

Nucleic Acid Assays

The DNA may be obtained from any cell source. DNA is extracted from the cell source or body fluid using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4 x 10⁹ base pairs).

In another alternate embodiment, RNA is isolated from biopsy tissue using standard methods well known to those of ordinary skill in the art such as guanidium thiocyanate-phenol-chloroform extraction (Chomocyznski et al., Anal. Biochem., 162:156, 1987). The isolated RNA is then subjected to coupled reverse transcription and amplification by polymerase chain reaction (RT-PCR), using specific oligonucleotide primers that are specific for a selected site. Conditions for primer annealing are chosen to ensure specific reverse transcription and amplification; thus, the appearance of an amplification product is diagnostic of the presence of a particular genetic variation. In another embodiment, RNA is reverse-transcribed and amplified, after which the amplified sequences are identified by, e.g., direct sequencing. In still another embodiment, cDNA obtained from the RNA can be cloned and sequenced to identify a mutation.

Protein Assays

In an alternate embodiment, biopsy tissue is obtained from a subject. Antibodies that are capable of specifically binding to mER are then contacted with samples of the tissue to determine the presence or absence of a mER polypeptide specified by the antibody. The antibodies may be polyclonal or monoclonal, preferably monoclonal. Measurement of specific antibody binding to cells may be accomplished by any known method, e.g., quantitative flow cytometry, enzyme-linked or fluorescence-linked immunoassay, Western analysis, etc.

Therapeutic Uses

According to the present invention, stimulation or inhibition of mER receptor activity may be used as a treatment option in patients with estrogen-related disease states. Alteration of mER receptor activity may be by methods, such as, but not limited to, (i) providing polypeptides that stimulate receptor activity, (ii) providing compounds that stimulate receptor activity, or (iii) providing compounds that inhibit receptor activity.

Gene Therapy

In a specific embodiment, vectors comprising a sequence encoding a protein, including, but not limited to, full-length mER, are provided to treat or prevent a disease or disorder associated with the function of mER. In this embodiment of the invention, the therapeutic vector encodes a sequence that produces the protein of the invention.

Any of the methods for gene therapy available in the art can be used according to the present invention. Exemplary methods are described below.

For general reviews of the methods of gene therapy, see, Goldspiel et al., Clinical Pharmacy, 1993, 12:488-505; Wu and Wu, Biotherapy, 1991, 3:87-95; Tolstoshev, Ann. Rev. Pharmacol. Toxicol., 1993, 32:573-596; Mulligan, Science, 1993, 260:926-932; and Morgan and Anderson, Ann. Rev. Biochem., 1993, 62:191-217; May, TIBTECH, 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses a protein of the invention in a suitable host. In particular, such a vector has a promoter operationally linked to the coding sequence for the protein. The promoter can be inducible or constitutive and, optionally, tissue-specific. In another embodiment, a nucleic acid molecule is used in which the protein coding sequences and any other desired sequences are flanked by regions that promote homologous recombination at a desired site in the genome, thus providing for intrachromosomal expression of the protein (Koller and Smithies, Proc. Natl. Acad. Sci. U.S.A, 1989, 86:8932-8935; Zijlstra et al., Nature, 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly provided in vivo, where it enters the cells of the organism and mediates expression of the protein. This can be accomplished by any of numerous methods known in the art, by constructing it as part of an appropriate expression vector and administering it so that it becomes intracellular, e.g., by infection using a defective or attenuated retroviral or other viral vector (see, U.S. Patent No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-S-1-64-N-acetylglucosamine polysaccharide; see, U.S. Patent No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem., 1987, 62:4429-4432), etc. In another embodiment, a nucleic acid ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Alternatively, the nucleic acid can be introduced intracellularly and incorporated within host cell DNA for expression by homologous recombination (Koller and Smithies, Proc. Natl. Acad. Sci. USA, 1989, 86:8932-8935; Zijlstra, et al., Nature, 1989, 342:435-438). These methods are in addition to those discussed above in conjunction with "Viral and Non-viral Vectors".

The form and amount of therapeutic nucleic acid envisioned for use depends on the type of disease and the severity of the desired effect, patient state, etc., and can be determined by one skilled in the art.

Inhibition or stimulation of protein synthesis

Gene transcription and protein translation may be inhibited or stimulated by administration of exogenous compounds. Exogenous compounds may interact with extracellular and/or intracellular messenger systems, such as, but not limited to, adenosine triphosphate, nitric oxide, guanosine triphosphate, and ion concentration; to regulate protein synthesis. In this embodiment, exogenous compounds that stimulate or inhibit mER protein synthesis may be used in the prevention and/or treatment for neurodegeneration, cardiovascular disease, infertility, and osteoporosis.

The present invention provides antisense nucleic acids (including ribozymes), which may be used to inhibit expression of mER of the invention. The antisense nucleic acid, upon hybridizing under cytoplasmic conditions with complementary bases in an RNA or DNA molecule, inhibits the role of the RNA or DNA. Additionally, hybridization of the antisense nucleic acid to the DNA or RNA may inhibit transcription of the DNA into RNA and/or translation of the RNA into the protein. If the RNA is a messenger RNA transcript, the antisense nucleic acid is a countertranscript or mRNA-interfering complementary nucleic acid. Antisense nucleic acid molecules can be encoded by a recombinant gene for expression in a cell (e.g., U.S. Patent No. 5,814,500; U.S. Patent No. 5,811,234) or can be prepared synthetically (e.g., U.S. Patent No. 5,780,607).

Alternatively, antibody molecules can also be administered, for example, by expressing nucleotide sequences encoding single-chain antibodies within the target cell population by utilizing, for example, techniques such as those described in Marasco et al. (Proc. Natl. Acad Sci. USA, 1993, 90:7889-7893).

The present invention also provides for an active agent which may be used to stimulate expression of mER of the invention. The active agent may interact with proteins present in cellular membrane to upregulate transcription by regulation of intracellular second messengers and transcription factors.

Therapeutically suggested compounds may be provided to the patient in formulations that are known in the art and may include any pharmaceutically acceptable additives, such as excipients, lubricants, diluents, flavorants, colorants, and disintegrants. The formulations may be produced in useful dosage units such as tablet, caplet, capsule, liquid, or injection.

The form and amount of therapeutic compound envisioned for use depends on the type of disease and the severity of the desired effect, patient state, etc., and can be determined by one skilled in the art.

EXAMPLES

The present invention will be better understood by reference to the following Examples, which are provided as exemplary of the invention, and not by way of limitation.

Materials and Methods

Chemicals

17β-Estradiol, Tamoxifen, and 17α ethynyl estradiol were obtained from Sigma Chemical Co. (St. Louis, MO). Genistein was obtained from Research Biochemical Inc. (Natick, MA). ICI 182,780 was obtained from Zeneca Pharmaceuticals (Mereside Alderley Park, Maccleffield Cheshire, England). Raloxifene was prepared using standard chemical procedures and techniques (U.S. Patent No. 4,418,068 and 6,080,762).

Isolation and cell culture of D12 cells

The D12 cell line was subcloned from an immortalized rat (E18) hypothalamic cell line (Fitzpatrick et al., Endocrinology, 140, 3928, 1999) obtained from Richard Robbins (Yale University). Immunocytochemical characterization of this cell line was performed with markers for endothelial cells (von Willebrand Factor 8 and DiI-ac-LDL), neurons (neurofilament M, NEU-N), astrocytes (GFAP), and fibroblasts (fibronectin). The predominant cell type in this cell line are endothelial cells with a small population (10%) staining positive for neurons.

D12 cells were grown at 37^{"}C in a humidified chamber with 5% CO₂ in Dulbecco=s Modified Eagle=s Medium (DMEM):F12 (1:1) (GIBCO-BRL, Gaithersburg, MD) supplemented with 5% fetal calf serum (GIBCO-BRL), 1% (v/v) penicillin (GIBCO-BRL), and 1% (v/v) GlutaMAX-1 (GIBCO-BRL). For membrane preparations, cells were plated in 150 mm² culture dishes at 3 x10⁶ cells/plate and the next day washed out with phenol red free DMEM:F12 media containing 5% charcoal stripped fetal calf serum (HyClone). On the third day cells were harvested for assay. For calcium mobilization experiments, cells were plated at 20,000 cells/glass coverslip and the following day the media exchanged to phenol red free stripped serum. After the 24 h washout of phenol the cells were loaded with a calcium indicator for calcium mobilization assays.

Fluorescence immunocytochemistry

D12 cells were washed in DPBS and lightly fixed for 30 min at RT in fixative containing 2% (v/v) paraformaldehyde, 0.15 M sucrose and 0.1% (v/v) glutaraldehyde in PBS (pH=7.4). Following fixation, cells were washed in DPBS and incubated for 1 h in 50 mM NH₄Cl and then blocked in 10% (v/v) bovine serum albumin (BSA) for 1 hr. Cells were incubated with antibody against ERα (MC-20 or H184) and caveolin-1 (C37120, Transduction Laboratories) for 3 h at room temperature then washed in DPBS and incubated with FITC (Jackson ImmunoResearch Laboratory, West Grove, PA)- and TRITC-labeled (Jackson ImmunoResearch Laboratory, West Grove, PA)-secondary antibodies for 1 h at room temperature. Cells were subsequently washed in DPBS and digitized images were obtained by fluorescent microscopy (Nikon PM2000).

Membrane fractionation

Initial experiments were done using sucrose gradients to identify plasma membranes from D12 cells. Briefly, cells were harvested in a binding buffer (10 mM Tris-HCl, 1 mM EDTA, 1mM DTT; pH=7.2) containing 5 μg/ml protease inhibitors (aprotinin, leupeptin, phosphoramidon, PMSF, pepstatin), pelleted to remove cell media and then homogenized by mechanical disruption (polytron; speed 6 for 10 sec). Unlysed cells and debris were removed by centrifugation at 15,000 x g for 15 min at 4ΕC. The resulting supernatant was homogenized and membranes were isolated by centrifugation at 100,000 x g for 1h at 4ΕC. The supernatant obtained following the high speed spin was labeled S2 (cytosol) while the pellet was labeled P2 (membranes). The pellet (P2) was resuspended using a glass homogenizer in 3 ml of 0.25 M sucrose in binding buffer. The sucrose gradient was layered in a 15 ml centrifuge tube starting with 41%, 25%, and 10% sucrose using J tubes. The P2 sample was added to the top and the remaining layer was capped with binding buffer. The tubes were centrifuged at 35,000 x g for 1 h, placed in a fraction collector (bottom tube draw) and 500 μl samples were collected. Protein concentrations were determined with BCA reagent (Pierce).

Radioligand binding assays

Equilibrium binding assays were performed with D12 extracts (40-60 (P2) or 10-20 (S2) _g protein /reaction) incubated with 10-600 pM of [¹²⁵I]-16-α-iodo-3,17-_-estradiol (NEN) for 2 h at room temperature. Unbound ligand was removed either by charcoal precipitation (soluble ER) or centrifugation (mER). For competition experiments, cold competitors (10⁻¹²-10⁶ M) were directly added to membranes and the binding reaction was initiated by adding 200 pM [¹²⁵I]-16-α-iodo-E2. A customized SAS-excel (SAS Institute, Cary, NC) application was written using a four parameter logistic model to determine IC50 values. A logistic dose transformation was performed on CPMs. Total bound CPMs and non-specific bound CPMs were used in the analysis as the maximum and minimum of the competition curves, respectively. For compounds repeated over several days, the IC50s were weighted by their respective standard errors (S.E.) to obtain an average IC50 and a confidence interval using a customized JMP (SAS Institute, Cary, NC) application. Statistical significance between IC50s and K_{D}s was determined using a pair-wise Z-test. The customized JMP applications were developed by Biometrics Research (Wyeth-Ayerst, Princeton, NJ).

Western blot analysis

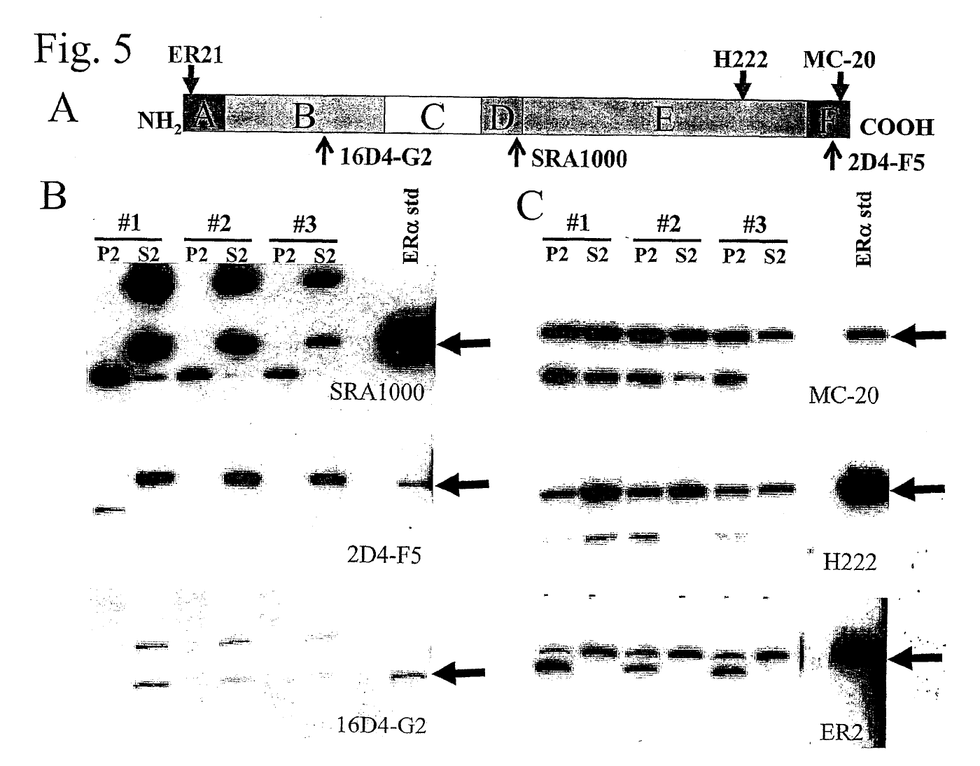

Cytosolic (S2) and membrane (P2) extracts were evaluated for ER expression by Western blots with a variety of antibodies generated against different epitopes of the ERα protein (Fig. 5). Equivalent amounts of protein or E2 binding activity (based on radioligand binding analyses) were fractionated by size on a 10% SDS-PAGE gel and then transferred to PVDF membranes for immunoblotting. Membranes were blocked for 1 hr at room temperature with blocking buffer (PBS, 5% milk and 0.03% (v/v) Tween-20) and then incubated with the primary antibody in blocking buffer overnight at 4^{"}C. The various ERα antibodies included H-184 (diluted 1:1000; SantaCruz Biotechnology, Inc); ER-21(diluted 1:1000; Blaustein, Endocrinology, 132, 1218, 1993); H222 (1:500; Greene et al. J. Steroid Biochem., 20, 51, 1984); 16D4-G2, 2D4-F5, 3E6-F2, and 8A11-F6 (all diluted 1:1000; Covance Research Products), SRA-1000 (diluted 1:1000; StressGen Biotechnologies Corp), 7A9-E1 (diluted 1:1000; generated by Wyeth) and MC-20 (diluted 1:2500; SantaCruz Biotechnology, Inc). Blots were washed the following morning in TPBS (PBS containing 0.3% (v/v) Tween-20) and incubated at room temperature for 2 hrs with a 1/20,000 dilution of the appropriate secondary antibody conjugated with HRP (Bio-Rad Laboratories). Blots were washed in TPBS, PBS, and immunoreactive bands were visualized with the SuperSignal chemiluminescent substrate (Pierce). Molecular mass standards (Amersham) and purified recombinant human ERα were included in each gel.

Calcium mobilization assay

D12 cells plated on glass coverslips were incubated for 30 min at 37ΕC in loading media (phenol red free DMEM high glucose, 0.1% (v/v) BSA and 10 _M sulfinpyrazone) containing 1 μM FURA2 A/M dispersed in pluronic acid (Molecular Probes, Eugene, OR). After loading the coverslips were rinsed in 2x volume of loading media and then equilibrated in 2x volume of HBS media (120 mM NaCl, 4.75 mM KCl, 1 mM KH₂PO₄, 1.44 mM MgSO₄, 5 mM NaHCO₃, 5.5 mM glucose, 20 mM HEPES; (pH=7.4)). Calcium recordings were performed using a fluorimeter (LS50B Perkin Elmer, Norwalk, CT) with excitation set at 340 channel 1 and 380 channel 2 with fixed emission set at a 509 wavelength. Analysis was done using FL WinLab version 3.0 software (Perkin Elmer, Norwalk, CT) with calibrations being performed using ionomycin (100 nM) for Rₘₐₓ and 5 mM EGTA for Rₘᵢₙ. Ratio data collection was done by first establishing a 2 min baseline followed by the addition of E2 (100 nM) directly into the HBS media time recording done for 15 min. The concentration of intracellular calcium was determined based on the Rₘₐₓ and Rₘᵢₙ determined in the calibration run.

Results

Immunocytochemistry

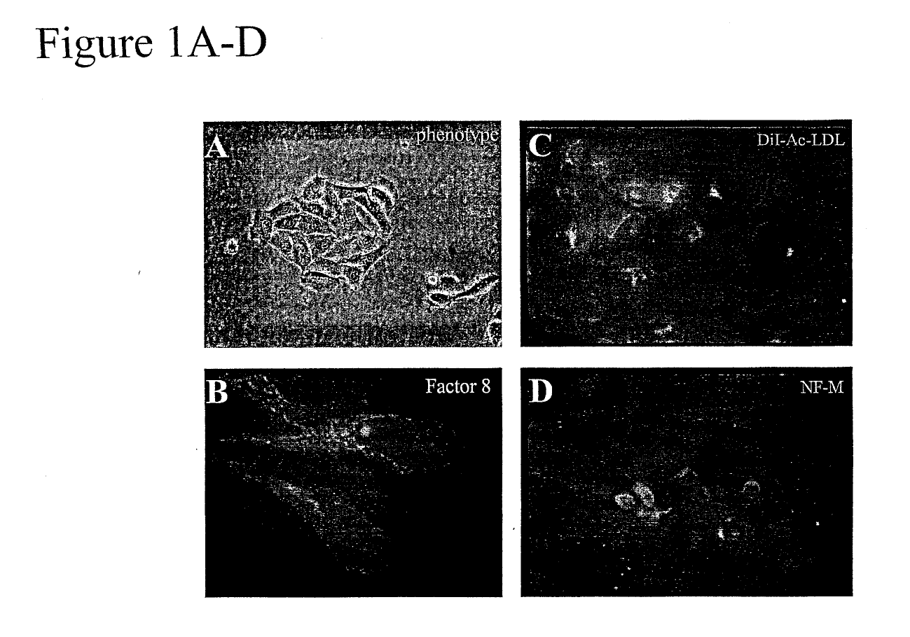

D12 cells exhibit multiple morphologies in culture suggesting they can differentiate into various cell types. One of the most prominent morphologies is cell clusters that resemble a Acobblestone matrix@ (phase contrast micrograph) (Fig. 1A). Immunocytochemical characterization of cultures indicate that the majority of cells are endothelial (>90%) based on staining with von Willebrand factor (Fig. 1B) and DiI-Ac-LDL uptake (Fig. 1C). A small subpopulation of cells in these cultures (<10%) appear to be neurons based on staining for the cytoskeletal marker neurofilament M (NF-M) (Fig. 1D). Cells within D12 cultures also stained positive with ER antibodies which is consistent with previous results indicating that this cell line expresses ERα_

Membrane vs nuclear ER isolation

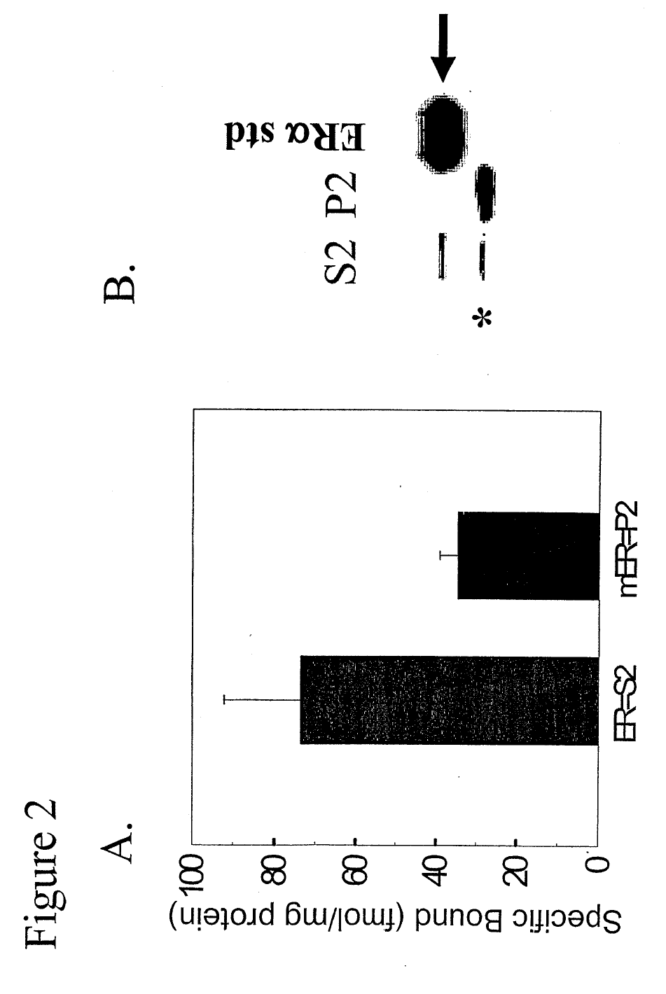

D12 cells were fractionated into cytosolic (S2) and membrane (P2) extracts by differential centrifugation and then analyzed for E2 binding activity by radioligand binding assays. Binding assays revealed specific binding activity in both membrane (P2) and cytosolic (S2) fractions (Fig. 2A). To ensure that the binding activity detected in P2 fractions was specific for membranes and not a result of contamination from S2, Western blot analysis was conducted on S2 and P2 fractions using a commercial antibody specific for ERα, SRA1000. This antibody detected a protein of about 67 kDa in the S2 fraction, whereas no band was identified in the P2 fraction (Fig. 2B). However, a protein band of about 55 kDa was found to cross-react with the antibody in both the S2 and P2 fraction (Fig. 2B). The amount of protein loaded for S2 and P2 samples were based on binding activity from the radioligand binding assays.

Scatchard analysis of ER in membranes vs cytosolic fraction

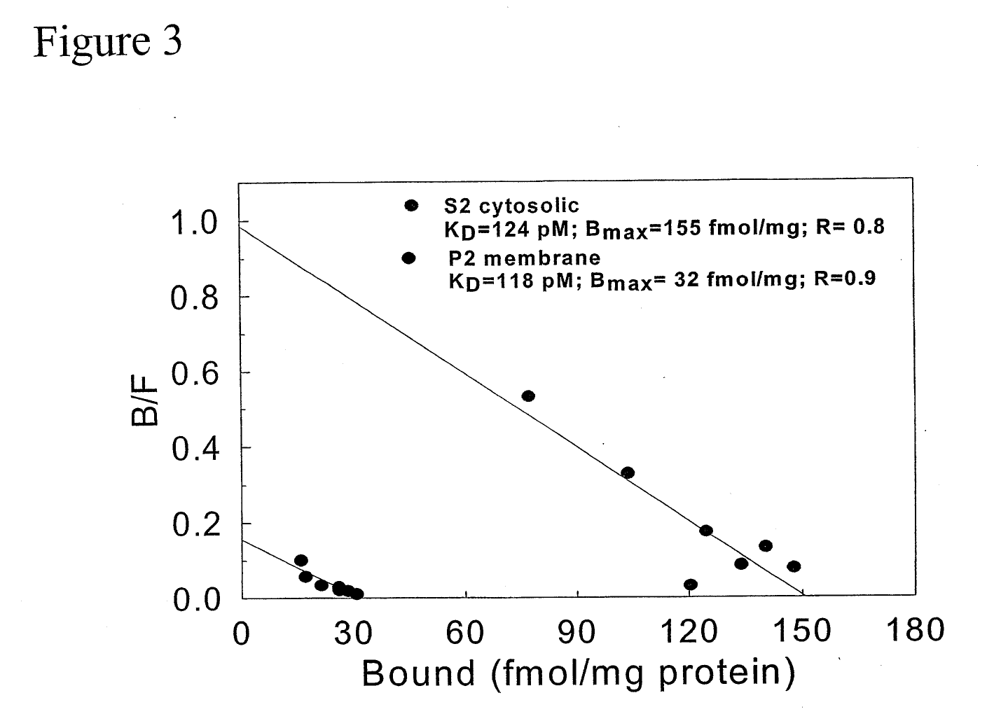

To compare the presence of ER compared with mER in D12 cells, membrane or cytosolic preparations were incubated with increasing amounts of [¹²⁵I]-16α-iodo-3,17β-E2 in the absence and presence of excess unlabeled 17β-E2 (1 _M). Specific, saturable binding sites were observed (Fig. 3). Scatchard analysis revealed a single high affinity binding site for the P2 and S2 fraction with a slope values of 0.9 and 0.8, respectively. Hence, binding parameters were determined using a locked slope of 1 as indicated in Materials and Methods. Linear regression of the data calculated K_{D} values of 118 ∀ 43.6 pM and a Bmax values of 32 ∀ 2.5 fmol/mg protein for membrane (P2) binding vs 124 ∀ 17.1pM and a Bmax of 187 ∀ 32.2 fmol/mg protein for cytosolic (S2) binding.

Selectivity of estrogen for the receptor labeling (membrane vs nuclear)

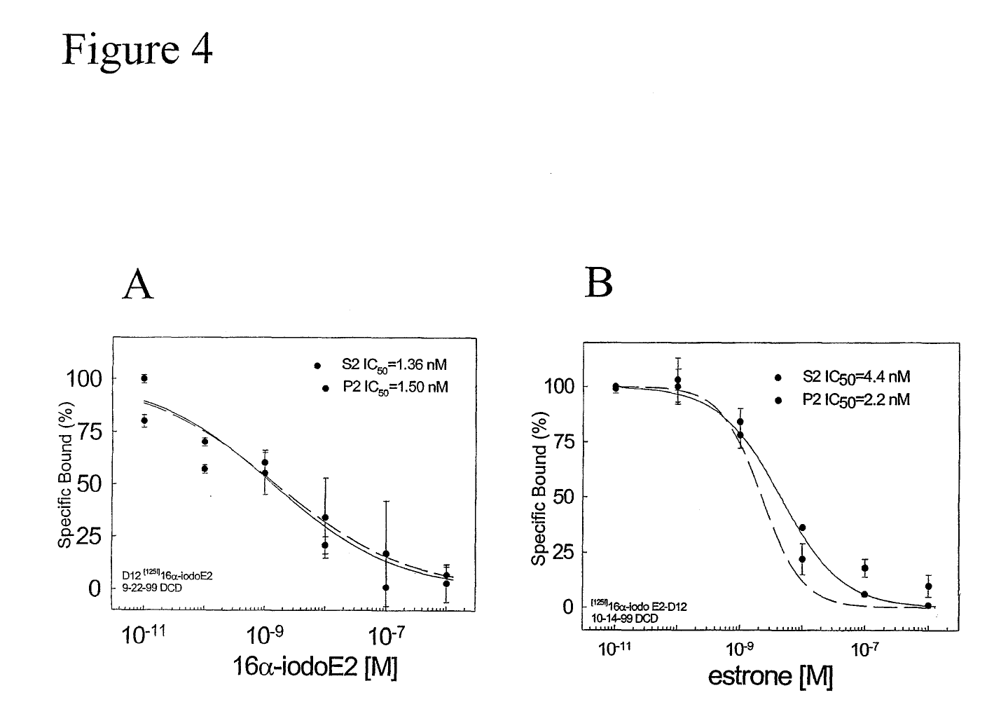

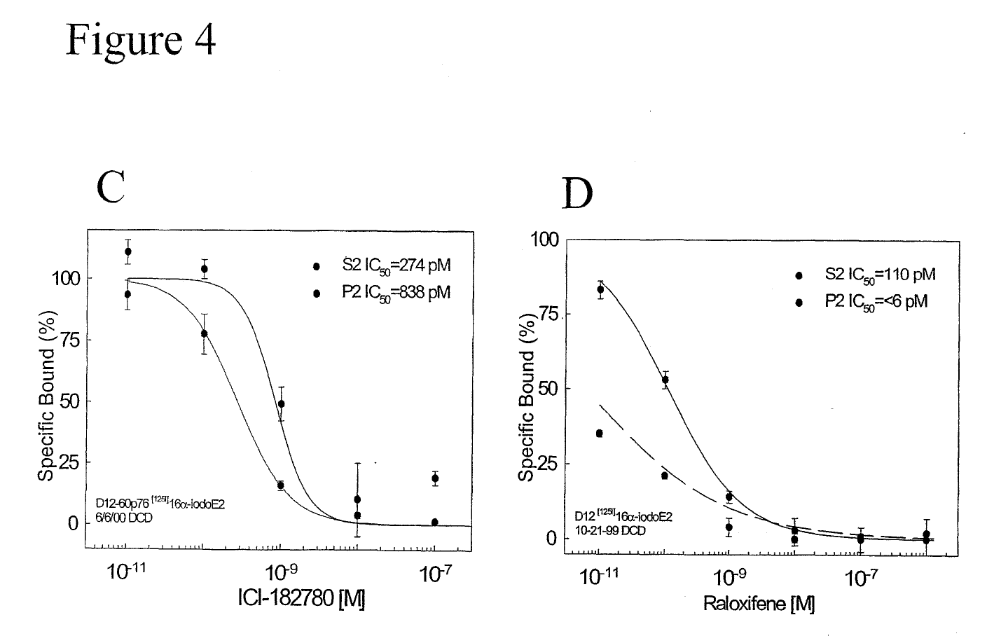

Various neurosteroids were competed for either the P2 or S2 fractions to determine selectivity of steroid interactions with the mER. Specificity was shown only for estrogens (Table 1) indicating that this protein is specific to estrogen action. Competition assays using [¹²⁵I]-16α-iodo-E2 were performed with a variety of known estrogen ligands. Estrone and unlabeled 16α-iodo-E2 (Fig. 4A and B) bound with similar IC50s whereas ICI-182780 and raloxifene showed differences in binding affinities (Fig. 4C and D). Additionally, IC50s values could not be determined for E2 when competing for the mER as the dose response curve was non-sigmodial whereas the IC50 value for the S2 was 0.1 nM (data not shown).

Table 1.

| | | |
|---|---|---|
| Ligand | ER (S2) | mER (P2) |
| 17-β-estradiol | + | + |
| Diethylstilbestrol (synthetic estrogen) | + | + |
| BPEA (anti-estrogen site) | - | - |
| Dihydrotestosterone | - | - |
| Dexmethasone | - | - |
| DHEA | - | - |
| Progesterone | - | - |
| Allopregnenolone | - | - |

All compounds tested at a concentration of 100 nM

Antibody recognition and differences

Pharmacological characterization of S2 and P2 extracts indicated that the E2 binding activity in D12 membranes had the properties of a receptor (saturable, selective, and reversible) that had distinguishing pharmacology from the nuclear ER. To gain a better understanding of the similarity of these two receptors at the amino acid level, D12 extracts were analyzed by Western blots using antibodies that recognized different epitopes along ERα (Fig. 5A). While all of the antibodies recognized the appropriate 67 kDa ER protein in S2 extracts, a subset of these antibodies also recognized a similar sized protein in P2 extracts (Fig. 5B and C). Of the 10 different ERα antibodies assayed by Western blots, only 4 were able to recognize a 67 kDa protein in both S2 and P2 fractions (Table 2). Western blot analysis of S2 and P2 fractions with a polyclonal antibody generated against ERβ did not reveal any staining (data not shown).

Table 2.

| Antibody | ERα Epitope | Domain | D12-S2 | D12-P2 |
|---|---|---|---|---|
| ER21 | 1-21 | A | + | + |
| H-184 | 2-185 | A/B | + | + |
| 3E6-F2 | 22-43 | A/B | + | - |
| 16D4-G2 | 127-141 | B | + | - |
| 8A11-F6 | 148-169 | B | + | - |
| SRA1000 | 287-300 | D | + | - |
| H222 | 463-528 | E | + | + |
| 7A9-E1 | 575-589 | F | + | - |
| 2D4-F5 | 575-595 | F | + | - |
| MC-20 | 580-599 | F | + | + |

Pharmacology of mER in presence of ERα antibody

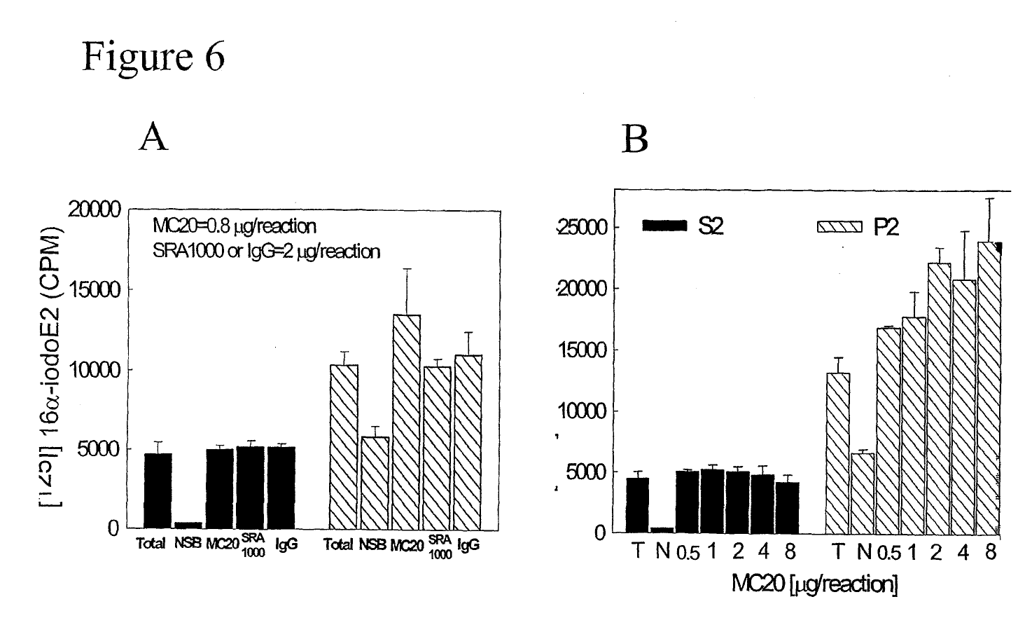

Specificity of the MC20 antibody for the membrane associated estrogen receptor was confirmed by Western blot analysis. Radioligand binding assays were use to determine whether the MC20 or SRA1000 would interfere with the ability of mER to bind a ligand. SRA1000 showed no interaction of mER labeling whereas MC20 statistically enhanced the labeling efficiency of [¹²⁵I] 16α-iodo-estradiol (Figure 6A). Additional studies indicated that the effect produced by MC-20 was dose-dependent (Figure 6B). This data provides additional evidence that MC20 can be used to label mER.

Immunocytochemical fluorescent staining of D12 cells

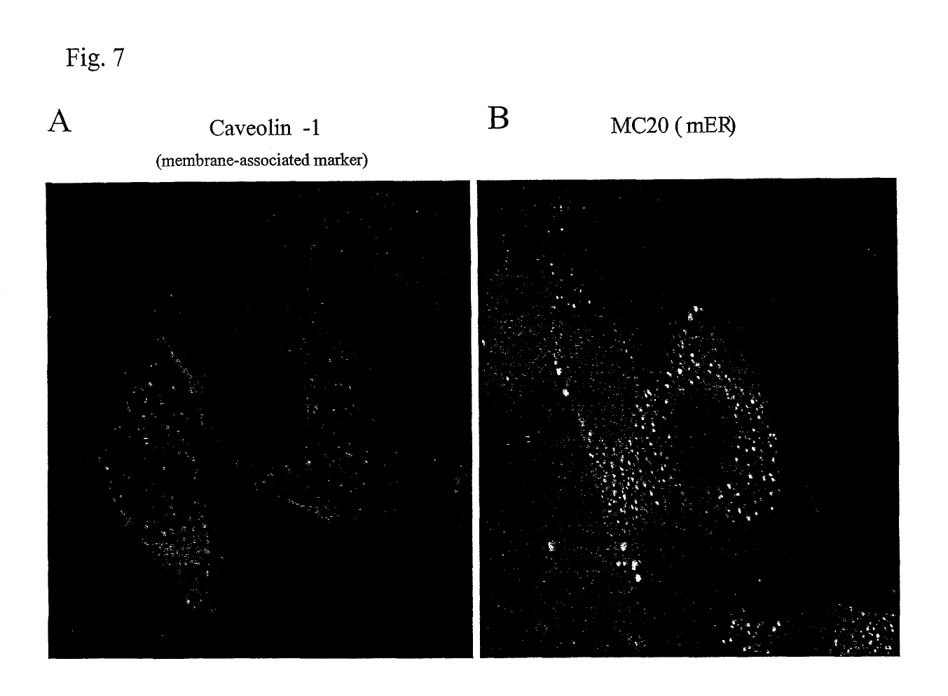

Cells were processed in a manner designed to preserve plasma membrane integrity and therefore minimize nuclear staining for ERα. Verification of MC20 antibody membrane labeling was done using immunocytochemistry. Light fixation of D12 cells and staining with MC20 antibody identified specific punctate labeling of the plasma membrane. The labeling pattern was similar to that observed for the membrane protein caveolin-1 (Figure 7A and 7B). This localization study using immunocytochemistry supports are finding that MC20 identifies a membrane bound estrogen receptor.

Calcium mobilization

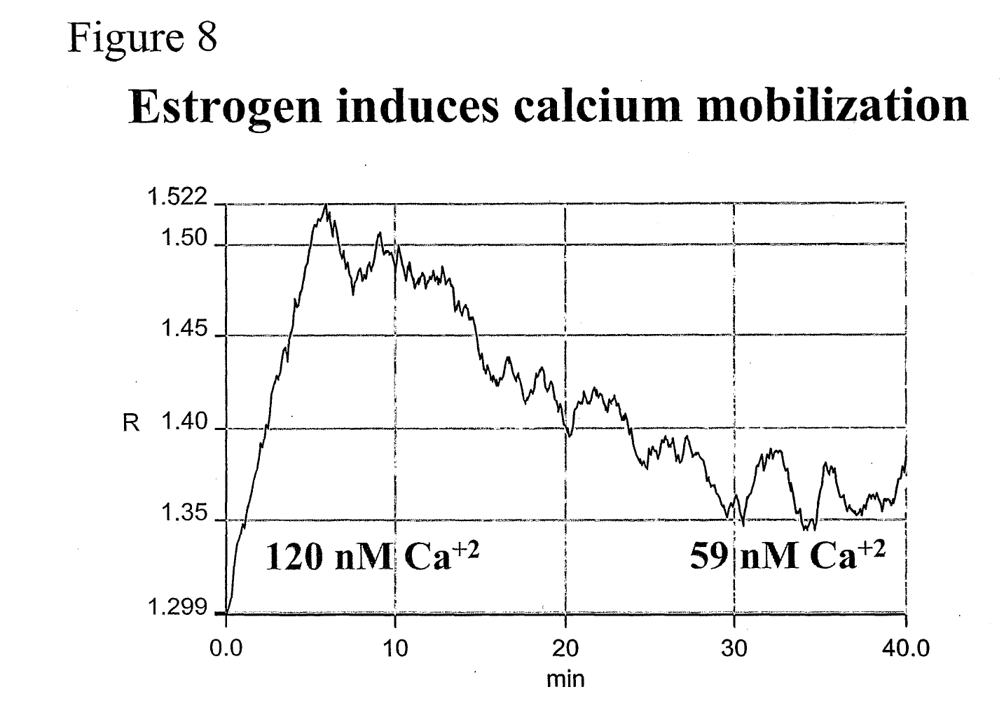

Labeling of a calcium indicator FURA 2A/M assisted in the identification of rapid calcium mobilization in the presence of estrogen (Figure 8). This rapid action of estrogen noted is proposed to occur through a membrane associated estrogen selective receptor.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

1. An isolated membrane estrogen receptor polypeptide, which membrane estrogen receptor polypeptide is present in a cellular P2 fraction, binds to an antibody specific for a nuclear ERα receptor antibody, and binds specifically to an estrogen compound.

2. The membrane estrogen receptor polypeptide of claim 1 wherein the estrogen compound is 17-β-estradiol or diethylstilbestrol.

3. The membrane receptor polypeptide of claim 1 or a fragment thereof, wherein the polypeptide or fragment has an apparent molecular weight of 67 kDa as determined by SDS-PAGE.

4. The membrane estrogen receptor polypeptide of claim 1 wherein the antibody is selected from the group consisting of ER21, H-184, H222, and MC-20.

5. The membrane estrogen receptor polypeptide of claim 1 wherein the receptor polypeptide is recognized by each of antibodies ER21, H-184, H222, and MC-20.

6. The membrane estrogen receptor polypeptide of claim 1 wherein the polypeptide is not recognized by nuclear ERα receptor antibody SRA1000.

7. The membrane estrogen receptor polypeptide of claim 1 wherein the membrane estrogen receptor polypeptide is not present in a cellular S2 fraction.

8. The membrane estrogen receptor polypeptide of claim 1 wherein binding of an estrogen compound to the receptor modulates calcium mobilization in a cell expressing the receptor.

9. An isolated membrane estrogen receptor polypeptide, which membrane estrogen receptor polypeptide is present in a cellular P2 fraction, binds to the nuclear ERα receptor antibodies ER21, H-184, H222, and MC-20, binds specifically to an estrogen compound, has an apparent molecular weight of 67 kDa, is not recognized by the nuclear ERα receptor antibody SRA1000 and is not present in the cellular S2 fraction.

10. A method for detecting a membrane estrogen receptor polypeptide, which method comprises detecting binding of a nuclear ERα receptor antibody to a polypeptide present in a membrane of a cell, wherein detection of such binding indicates the presence of a membrane estrogen receptor.

11. The method according to claim 10, wherein the membrane estrogen receptor is detected in a P2 cellular fraction.

12. The method according to claim 10, wherein the membrane estrogen receptor is detected in an intact cell.

13. The method of claim 10, wherein the nuclear ERα receptor antibody is selected from the group consisting of ER21, H-184, H222, and MC-20.

14. A method for detecting the membrane estrogen receptor polypeptide of claim 1, which method comprises detecting binding of an estrogen compound to a polypeptide in a sample containing the P2 cellular fraction, wherein detection of such binding indicates the presence of a membrane estrogen receptor polypeptide.

15. The method of claim 14 wherein the estrogen compound is 17-β-estradiol or diethylstilbestrol.

16. A method for identifying a compound that binds the membrane estrogen receptor of claim 1, which method comprises detecting binding of a test compound contacted with a cellular P2 fraction comprising a membrane estrogen receptor wherein binding of the test compound indicates that the test compound binds to the membrane estrogen receptor.

17. The method according to claim 16, wherein detection of binding of the test compound comprises detecting inhibition of binding of an estrogen compound to the cellular P2 fraction.

18. A method for identifying a compound that modulates the membrane estrogen receptor of claim 1, which method comprises detecting calcium mobilization in a cell comprising a membrane estrogen receptor contacted with a test compound, wherein mobilization of calcium indicates that the test compound binds the membrane estrogen receptor.

19. The method according to claim 18, which further comprises detecting genomic estrogen receptor activity; wherein alteration of genomic activity in the presence of the test compound indicates that the compound does not selectively modulate the membrane estrogen receptor.

20. A method of screening for an antagonist of the membrane estrogen receptor polypeptide of claim 1, which method comprises (i) contacting a cell that expresses the membrane estrogen receptor polypeptide of claim 1 with a test compound and an estrogen compound and (ii) detecting decreased calcium mobilization compared to contacting the cell with the estrogen compound alone.
